(19) United States
(12) Patent Application Publication
(10) Pub. No.: US 2020/0166517 A1
(43) Pub. Date: May 28, 2020

(54) Screening Method for Identification of Neuroprotective Compounds

(71) Applicants: Temple University-Of The Commonwealth System of Higher Education, Philadelphia, PA (US); Shriners Hospitals for Children, Tampa, FL (US)

(72) Inventors: Gareth M. Thomas, Philadelphia, PA (US); Dale D.O. Martin, Philadelphia, PA (US); Sabrina Marion Holland, Philadelphia, PA (US); Jingwen Niu, Wallingford, PA (US); Marlene A. Jacobson, Melrose Park, PA (US); Wayne E. Childers, New Hope, PA (US)

(21) Appl. No.: 16/631,969

(22) PCT Filed: Jul. 18, 2018
(86) PCT No.: PCT/US18/42620

§ 371 Date: Jan. 17, 2020

Related U.S. Application Data
(60) Provisional application No. 62/534,347, filed on Jul. 19, 2017.

(51) Int. Cl. G01N 33/58; G01N 33/68; G01N 33/573

(52) U.S. Cl. CPC G01N 33/582; G01N 2800/28; G01N 33/573; G01N 33/6896

(57) ABSTRACT

The present invention provides compositions and methods for identifying modulators of palmitoylation and uses thereof.

### CROSS-REFERENCE TO RELATED APPLICATIONS

This application claims priority from U.S. Provisional Application Ser. No. 62/534,347, filed Jul. 19, 2017, the contents of which are incorporated by reference herein in its entirety.

### BACKGROUND OF THE INVENTION

The c-Jun N-terminal Kinase (JNK) pathway plays a critical role in several forms of neuronal degeneration and death. Based on this knowledge, numerous efforts have been made over the last 10-20 years to develop direct inhibitors of JNK as neuroprotectants. However, few JNK inhibitors have proved viable, perhaps because JNKs also play diverse physiological roles. More recently, attention has shifted to Dual Leucine-zipper Kinase (DLK), an upstream JNK pathway kinase that plays a specific role in injury- and stress-induced JNK signaling. Indeed, an inhibitor of DLK's kinase activity is currently in clinical trials for the neurodegenerative condition Amyotrophic Lateral Sclerosis (ALS). Unfortunately, though, direct inhibitors of DLK's kinase activity may suffer from a lack of specificity, limiting their therapeutic potential.

Thus, there is a need in the art for the identification of improved neuroprotective compositions.

### SUMMARY OF THE INVENTION

In one aspect, the invention provides a method of identifying a modulator of DLK palmitoylation comprising: administering at least one test compound to a cell modified to comprise DLK tagged with a detectable label, detecting DLK membrane association; and identifying the test compound as a modulator of palmitoylation when the test compound alters DLK membrane association.

In one embodiment, the test compound is identified as an inhibitor when DLK membrane association is reduced compared to control conditions where a test compound is not administered.

In one embodiment, the identified inhibitor is at least one selected from the group consisting of: a broad palmitoylation inhibitor, a specific inhibitor of one or more palmitoyl acyltransferase (PAT), and a modulator of DLK conformation.

In one embodiment, the method comprises the use of a high throughput screen and wherein the test compound is from a library of test compounds.

In one embodiment, DLK membrane association is detected by quantifying puncta of the detectable label.

In one embodiment, the method further comprises conducting a cytotoxicity assay on an identified inhibitor of palmitoylation.

In one embodiment, the method further comprises conducting an assay to evaluate the effect of the identified inhibitor of palmitoylation on neurodegeneration.

In one embodiment, the identified inhibitor of palmitoylation is neuroprotective.

In one aspect, the invention provides a method of treating a disease or disorder in a subject in need thereof, comprising administering an effective amount of a modulator of palmitoylation.

In one embodiment, the disease or disorder is associated with neurodegeneration.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1A and FIG. 1B demonstrate that DLK's Role in Neurodegeneration is Likely Due to its Unique Palmitoylation. FIG. 1A: DLK conveys pathological signals following insult or injury, predominantly to JNK2/JNK3. In contrast, physiological signaling is controlled by other MAP3Ks that mainly signal via JNK1. FIG. 1B: DLK, but not homologous MAP3Ks, is covalently modified with the lipid palmitate (orange lipid). (Holland, 2016).

FIG. 2A and FIG. 2B demonstrate that DLK must be palmitoylated to activate JNK3. FIG. 2A: HEK293T cells expressing myc-JNK3 plus wtDLK-GFP were treated with the palmitoylation inhibitor 2-Bromopalmitate (2-Br) or EtOH (vehicle). 2-Br blocks JNK3 activation by wtDLK-GFP, as detected by phosphoJNK antibody. FIG. 2B: HEK293T cells were transfected with myc-JNK3 plus either wtDLK-GFP or a DLK palmitoyl site mutant (DLK-CS-GFP). Only wtDLK-GFP activates JNK3, as detected by phosphoJNK antibody.

FIG. 3A and FIG. 3B demonstrate the dramatic palmitoylation-dependent Control of DLK Subcellular Localization. FIG. 3A: HEK293T cells were transfected with wtDLK-GFP or DLK-CS-GFP and treated 4 h later with 2-Br or vehicle. Top: Live images of GFP fluorescence, acquired 8 h post-transfection. Bottom: The same images, thresholded to an identical value to highlight membrane-localized wtDLK-GFP puncta (red), which are essentially absent in the 2-Br and DLK-CS-GFP conditions. FIG. 3B: A subset of cells from FIG. 3A were fixed and immunostained with anti-GFP antibody. Images of GFP fluorescence (n=5 per condition) were analyzed by ImageXpress software. Palmitoylation-dependent differences in number of DLK-GFP puncta are readily detectable.

FIG. 4 depicts an orthogonal assay to confirm mechanism of action (MOA) of HTS Hits. HEK293T cells were transfected with wt-DLK-GFP or DLK-CS-GFP and palmitoyl-proteins were isolated by Acyl-Biotin Exchange (ABE). Palmitoyl-DLK is robustly detected in ABE fractions but no ABE signal is seen from cells treated with 2-Br, or if DLK's palmitoyl-site is mutated. DLK expressed similarly in all conditions. (Holland, 2016)

FIG. 5A-FIG. 5D demonstrates that Palmitoyl-DLK is essential for trophic deprivation (TD) induced degeneration. FIG. 5A: Schematic of lentiviral vector expressing GFP (infectivity marker) with or without DLK shRNA. Virus made from this vector can be coinfected with virus expressing shRNA-resistant (shr) wtDLK or DLK-CS. FIG. 5B: Rat sensory neurons infected with the indicated viruses were lysed 7d later. DLK levels are greatly reduced by DLK shRNA and restored by rescue constructs. FIG. 5C: Images of embryonic rat sensory neurons infected with the indicated viruses at DIV0, subjected to TD at DIV5 and fixed 24 later to detect microtubules (Tuj1). TD-induced degeneration (seen as break-up of Tuj1 staining) is prevented by DLK knockdown. FIG. 5D: Degeneration index, calculated from 4 images per condition from FIG. 5C confirms widespread TD-induced degeneration in control virus-infected neurons, but not DLK ‘knockdown’ neurons. Shr-wtDLK, but not DLK-CS, rescues TD-induced degeneration, which thus critically requires palmitoyl-DLK.*; p<0.01; n.s., not significant.

FIG. 6A-FIG. 6C depicts the results of experimental example demonstrating that Golgi localization of DLK-GFP in HEK293T cells is specific and dependent on palmitoylation. FIG. 6A depicts HEK293T cells transiently expressing DLK-GFP, fixed and immunostained with antibodies to detect GFP and the Golgi marker GM130. FIG. 6B depicts HEK293T cells, transfected as in FIG. 6A to express either wild type DLK-GFP (DLK-GFP) or a DLK palmitoylation site mutant (C127S-DLK-GFP). C127S mutation, or treatment of DLK-GFP-expressing cells with the palmitoylation inhibitor 2BP diffuses the Golgi-associated clusters of DLK-GFP. FIG. 6C depicts results from an experiment in which HEK293T cells were seeded into 12 wells of a 96-well plate and transfected with DLK-GFP and then treated with 2BP or vehicle (6 wells per condition). Cells were fixed in PFA and imaged using an ImageXpress High Content Imaging system to detect GFP signal. Assay quality was determined by calculating the z-prime (z′) for 6 determinations for each of the indicated conditions (z′=S/R, S=[(Mean of Vehicle treated−3×SD)−(Mean of 2BP−3×SD)], R=Vehicle Mean−2BP mean).

FIG. 7A-FIG. 7C depicts the results of experimental example demonstrating a high content imaging screen identifies ketoconazole as the most potent compound to inhibit DLK-GFP puncta formation. FIG. 7A depicts HEK293T cells co-transfected with DLK-GFP plus mCherry-NLS×3 and then treated with 2BP or vehicle were fixed to detect GFP, mCherry and the nuclear marker DAPI. 2BP reduces DLK-GFP puncta without affecting mCherry-NLS×3 expression or DAPI signal. FIG. 7B depicts design of the high-throughput screen for compounds that inhibit DLK-GFP puncta formation. FIG. 7C depicts compounds from the Prestwick Chemical Library™, which were spotted onto 96 well plates at 10 mM per compound in DMSO and diluted in DMEM prior to adding to transfected cells in duplicate at a final concentration of 10 micromolar (similar to the flow chart in FIG. 7B). 16 h later, cells were fixed in 4% PFA and stained with DAPI. High-content imaging was performed using an ImageXPress Image Analysis ‘TransFluor’ and Multi-wavelength scoring (MWS) modules. Data are plotted as total number of puncta (TransFluor Module) per total NLS (MWS module) for each compound. Drugs that decreased the nuclear NLS and/or DAPI markers below 3 times the mean were excluded due to likely toxicity or non-specific effects (plotted in grey). ‘Hits’ were drugs characterised as decreasing all ‘puncta’ readouts by more than 3 times the SD and included in 2 replicates.

FIG. 8A-FIG. 8C depicts the results of experimental example demonstrating that Ketoconazole decreases DLK-GFP puncta and inhibits DLK palmitoylation in a dose dependent manner. FIG. 8A depicts an experiment in which HEK293T cells transiently transfected with DLK-GFP and mCherry-NLS and subsequently incubated with the indicated concentrations of Ketoconazole or 20 μM 2BP 2 hours post-transfection for 16-18 h. Cells were fixed and stained with DAPI. Scale bar represents 50 μm. FIG. 8B depicts quantified DLK puncta/NLS and nuclear counts (DAPI) from cells transfected as in FIG. 8A and treated with the indicated concentrations of ketoconazole. FIG. 8C depicts an experiment in which transiently transfected HEK293T cells were prepared as in FIG. 8A and palmitoylation (HAM+) was detected using the ABE assay. HAM- sample was generated from an equal fraction of all conditions combined.

FIG. 9A-FIG. 9C depicts the results of experimental example demonstrating that Ketoconazole inhibits palmitoylation of DLK and PSD-95, but not GAP43. HEK293T cells were transiently transfected with FIG. 9A) DLK-GFP, FIG. 9B) GAP43-Myc, FIG. 9C) and untagged PSD95 and incubated with 20 μM 2BP, or with 2.5 μM or 5 μM ketoconazole 2 h post-transfection for 16-18 h. Palmitoylation was detected using the ABE assay (left) and quantified from n=4 determinations per condition (right). HAM- includes an equal fraction of all conditions combined. Ketoconazole significantly reduces palmitoylation of DLK and PSD-95, but does not affect palmitoylation of GAP-43. One-way ANOVA, Kruskal-Wallis post-hoc analysis; FIG. 9A) ANOVA p=0.0214, h=7.692, FIG. 9B) ANOVA not significant, FIG. 9C) ANOVA p=0.0158, h=8.290. Error bars represent SEM.

FIG. 10A-FIG. 10B depicts the results of experimental example demonstrating that Ketoconazole significantly decreases DLK-mediated phospho-cJun activation in primary neurons. FIG. 10A depicts an experiment in which dorsal Root Ganglion (DRG) neurons were pretreated at 7 Days in vitro (DIV 7) with 2.5 μM Ketoconazole overnight or 20 μM 2BP for 2 h prior to a 2.5 h NGF withdrawal in presence of the indicated compound. Cells were lysed in SDS-PAGE loading buffer and levels of endogenous DLK, phospho-cJun and tubulin were detected by Western blot.

FIG. 10B depicts quantification of phospho-cJun normalised to -NGF vehicle treated cells. Two-way ANOVA indicates significant effects of interaction (p=0.0071), NGF (p=0.0026) and Ketoconazole (p=0.0001). The effect of Ketoconazole in DRGs undergoing NGF withdrawal was also significant as determined by the Bonferroni post-test (p<0.01). Error bars represent SEM.

### DETAILED DESCRIPTION

The present invention provides compositions and methods for the identification of regulators of Dual Leucine-zipper Kinase (DLK) palmitoylation. Further, the present invention provides methods of regulating DLK activity in a cell by administering to the cell one or more of the identified regulators. In certain embodiments, the invention provides a method of treating or preventing a disease or disorder associated with the activity of a palmitoylated protein, including, but not limited to, palmitoylated DLK. For example, in certain embodiments, the invention provides a method for treating or preventing a neurological or neurodegenerative disease or disorder, including but not limited to neurodegeneration following acute injury (e.g., stroke, traumatic brain injury, peripheral nerve injury), Alzheimer's disease, Amyotrophic Lateral Sclerosis (ALS), chemotherapy-induced peripheral neuropathy (CIPN), diabetic neuropathy, and HIV-associated neuropathies. In certain embodiments, the invention provides a method of treating or preventing certain cancers that are associated with aberrant palmitoylation.

DLK is an ‘executioner’ enzyme that controls degeneration or death of several types of neurons. DLK critically requires modification with the lipid palmitate, a process called palmitoylation, to perform this role. Described herein is a novel screening method to identify compounds that prevent DLK palmitoylation, and thereby act as neuroprotectants. Further, identified compounds can also be used for the study of neurodegenerative mechanisms, study of the role of palmitoylation in various biological processes, and for the treatment or protection of other diseases or disorders associated with aberrant palmitoylation.

### Definitions

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

A “modulator of palmitoylation” as used herein refers to any compound, biomolecule, small molecule, or the like that influences the amount, extent, or level of palmitoylation of a substrate. In certain embodiments, the modulator enhances or increases palmitoylation of a substrate. In certain embodiments, the modulator decreases or inhibits palmitoylation of a substrate.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

“Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences.

By the term “applicator,” as the term is used herein, is meant any device including, but not limited to, a hypodermic syringe, a pipette, and the like, for administering the compounds and compositions of the invention.

As used herein, “aptamer” refers to a small molecule that can bind specifically to another molecule. Aptamers are typically either polynucleotide- or peptide-based molecules. A polynucleotidal aptamer is a DNA or RNA molecule, usually comprising several strands of nucleic acids, that adopt highly specific three-dimensional conformation designed to have appropriate binding affinities and specificities towards specific target molecules, such as peptides, proteins, drugs, vitamins, among other organic and inorganic molecules. Such polynucleotidal aptamers can be selected from a vast population of random sequences through the use of systematic evolution of ligands by exponential enrichment. A peptide aptamer is typically a loop of about 10 to about 20 amino acids attached to a protein scaffold that bind to specific ligands. Peptide aptamers may be identified and isolated from combinatorial libraries, using methods such as the yeast two-hybrid system.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are substantially complementary to each other when at least about 50%, preferably at least about 60% and more preferably at least about 80% of corresponding positions in each of the molecules are occupied by nucleotides which normally base pair with each other (e.g., A:T and G:C nucleotide pairs).

A “disease” is a state of health of an animal wherein the animal cannot maintain homeostasis, and wherein if the disease is not ameliorated then the animal's health continues to deteriorate. In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

Signal transduction is any process by which a cell converts one signal or stimulus into another, most often involving ordered sequences of biochemical reactions carried out within the cell. The number of proteins and molecules participating in these events increases as the process eminates from the initial stimulus resulting in a “signal cascade.” The phrase “downstream effector”, as used herein, refers to a protein or molecule acted upon during a signaling cascade, which in term acts upon another protein or molecule. The term “downstream” indicates the direction of the signaling cascade.

A disease or disorder is “alleviated” if the severity of a symptom of the disease, or disorder, the frequency with which such a symptom is experienced by a patient, or both, are reduced.

The terms “effective amount” and “pharmaceutically effective amount” refer to a nontoxic but sufficient amount of an agent to provide the desired biological result. That result can be reduction and/or alleviation of the signs, symptoms, or causes of a disease or disorder, or any other desired alteration of a biological system. An appropriate effective amount in any individual case may be determined by one of ordinary skill in the art using routine experimentation.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

The term “expression vector” as used herein refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules, siRNA, ribozymes, and the like. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well.

As used herein, the term “fragment,” as applied to a nucleic acid, refers to a subsequence of a larger nucleic acid. A “fragment” of a nucleic acid can be at least about 15 nucleotides in length; for example, at least about 50 nucleotides to about 100 nucleotides; at least about 100 to about 500 nucleotides, at least about 500 to about 1000 nucleotides; at least about 1000 nucleotides to about 1500 nucleotides; about 1500 nucleotides to about 2500 nucleotides; or about 2500 nucleotides (and any integer value in between). As used herein, the term “fragment,” as applied to a protein or peptide, refers to a subsequence of a larger protein or peptide. A “fragment” of a protein or peptide can be at least about 20 amino acids in length; for example, at least about 50 amino acids in length; at least about 100 amino acids in length; at least about 200 amino acids in length; at least about 300 amino acids in length; or at least about 400 amino acids in length (and any integer value in between).

The term “fusion polypeptide” refers to a chimeric protein containing a protein of interest (e.g., luciferase) joined to a heterologous sequence (e.g., a non-luciferase amino acid or protein).

The term “homology” refers to a degree of complementarity. There may be partial homology or complete homology (i.e., identity). Homology is often measured using sequence analysis software (e.g., Sequence Analysis Software Package of the Genetics Computer Group. University of Wisconsin Biotechnology Center. 1710 University Avenue. Madison, Wis. 53705). Such software matches similar sequences by assigning degrees of homology to various substitutions, deletions, insertions, and other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

“Instructional material,” as that term is used herein, includes a publication, a recording, a diagram, or any other medium of expression which can be used to communicate the usefulness of the composition and/or compound of the invention in a kit. The instructional material of the kit may, for example, be affixed to a container that contains the compound and/or composition of the invention or be shipped together with a container which contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

The term “isolated” when used in relation to a nucleic acid, as in “isolated oligonucleotide” or “isolated polynucleotide” refers to a nucleic acid sequence that is identified and separated from at least one contaminant with which it is ordinarily associated in its source. Thus, an isolated nucleic acid is present in a form or setting that is different from that in which it is found in nature. In contrast, non-isolated nucleic acids (e.g., DNA and RNA) are found in the state they exist in nature. For example, a given DNA sequence (e.g., a gene) is found on the host cell chromosome in proximity to neighboring genes; RNA sequences (e.g., a specific mRNA sequence encoding a specific protein), are found in the cell as a mixture with numerous other mRNAs that encode a multitude of proteins. However, isolated nucleic acid includes, by way of example, such nucleic acid in cells ordinarily expressing that nucleic acid where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide contains at a minimum, the sense or coding strand (i.e., the oligonucleotide may be single-stranded), but may contain both the sense and anti-sense strands (i.e., the oligonucleotide may be double-stranded).

The term “isolated” when used in relation to a polypeptide, as in “isolated protein” or “isolated polypeptide” refers to a polypeptide that is identified and separated from at least one contaminant with which it is ordinarily associated in its source. Thus, an isolated polypeptide is present in a form or setting that is different from that in which it is found in nature. In contrast, non-isolated polypeptides (e.g., proteins and enzymes) are found in the state they exist in nature.

By the term “modulating,” as used herein, is meant mediating a detectable increase or decrease in the level of a mRNA, polypeptide, or a response in a subject compared with the level of a mRNA, polypeptide or a response in the subject in the absence of a treatment or compound, and/or compared with the level of a mRNA, polypeptide, or a response in an otherwise identical but untreated subject. The term encompasses perturbing and/or affecting a native signal or response thereby mediating a beneficial therapeutic response in a subject, preferably, a human.

“Naturally-occurring” as applied to an object refers to the fact that the object can be found in nature. For example, a polypeptide or polynucleotide sequence that is present in an organism (including viruses) that can be isolated from a source in nature and which has not been intentionally modified by man is a naturally-occurring sequence.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). The term “nucleic acid” typically refers to large polynucleotides.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

By “expression cassette” is meant a nucleic acid molecule comprising a coding sequence operably linked to promoter/regulatory sequences necessary for transcription and, optionally, translation of the coding sequence.

An “oligonucleotide” or “polynucleotide” is a nucleic acid ranging from at least 2, preferably at least 8, 15 or 25 nucleotides in length, but may be up to 50, 100, 1000, or 5000 nucleotides long or a compound that specifically hybridizes to a polynucleotide. Polynucleotides include sequences of deoxyribonucleic acid (DNA) or ribonucleic acid (RNA) or mimetics thereof which may be isolated from natural sources, recombinantly produced or artificially synthesized. A further example of a polynucleotide of the present invention may be a peptide nucleic acid (PNA). (See U.S. Pat. No. 6,156,501 which is hereby incorporated by reference in its entirety.) The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this disclosure. It will be understood that when a nucleotide sequence is represented herein by a DNA sequence (e.g., A, T, G, and C), this also includes the corresponding RNA sequence (e.g., A, U, G, C) in which “U” replaces “T”.

The term “operably linked” as used herein refer to the linkage of nucleic acid sequences in such a manner that a nucleic acid molecule capable of directing the transcription of a given gene and/or the synthesis of a desired protein molecule is produced. The term also refers to the linkage of sequences encoding amino acids in such a manner that a functional (e.g., enzymatically active, capable of binding to a binding partner, capable of inhibiting, etc.) protein or polypeptide is produced.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a n inducible manner.

An “inducible” promoter is a nucleotide sequence which, when operably linked with a polynucleotide which encodes or specifies a gene product, causes the gene product to be produced substantially only when an inducer which corresponds to the promoter is present.

“Polypeptide” refers to a polymer composed of amino acid residues, related naturally occurring structural variants, and synthetic non-naturally occurring analogs thereof linked via peptide bonds. Synthetic polypeptides can be synthesized, for example, using an automated polypeptide synthesizer.

The term “protein” typically refers to large polypeptides.

The term “peptide” typically refers to short polypeptides.

Conventional notation is used herein to portray polypeptide sequences: the left-hand end of a polypeptide sequence is the amino-terminus; the right-hand end of a polypeptide sequence is the carboxyl-terminus.

As used herein, a “peptidomimetic” is a compound containing non-peptidic structural elements that is capable of mimicking the biological action of a parent peptide. A peptidomimetic may or may not comprise peptide bonds.

A “polynucleotide” means a single strand or parallel and anti-parallel strands of a nucleic acid. Thus, a polynucleotide may be either a single-stranded or a double-stranded nucleic acid. In the context of the present invention, the following abbreviations for the commonly occurring nucleic acid bases are used. “A” refers to adenosine, “C” refers to cytidine, “G” refers to guanosine, “T” refers to thymidine, and “U” refers to uridine.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

The term “recombinant polypeptide” as used herein is defined as a polypeptide produced by using recombinant DNA methods. A host cell that comprises a recombinant polynucleotide is referred to as a “recombinant host cell.” A gene which is expressed in a recombinant host cell wherein the gene comprises a recombinant polynucleotide, produces a “recombinant polypeptide.”

As used herein, a “recombinant cell” is a host cell that comprises a recombinant polynucleotide.

“Ribozymes” as used herein are RNA molecules possessing the ability to specifically cleave other single-stranded RNA in a manner analogous to DNA restriction endonucleases. Through the modification of nucleotide sequences encoding these RNAs, molecules can be engineered to recognize specific nucleotide sequences in an RNA molecule and cleave it (Cech, 1988, J. Amer. Med. Assn. 260:3030). There are two basic types of ribozymes, namely, tetrahymena-type (Hasselhoff, 1988, Nature 334:585) and hammerhead-type. Tetrahymena-type ribozymes recognize sequences which are four bases in length, while hammerhead-type ribozymes recognize base sequences 11-18 bases in length. The longer the sequence, the greater the likelihood that the sequence will occur exclusively in the target mRNA species. Consequently, hammerhead-type ribozymes are preferable to tetrahymena-type ribozymes for inactivating specific mRNA species, and 18-base recognition sequences are preferable to shorter recognition sequences which may occur randomly within various unrelated mRNA molecules. Ribozymes and their use for inhibiting gene expression are also well known in the art (see, e.g., Cech et al., 1992, J. Biol. Chem. 267:17479-17482; Hampel et al., 1989, Biochemistry 28:4929-4933; Eckstein et al., International Publication No. WO 92/07065; Altman et al., U.S. Pat. No. 5,168,053).

By the term “specifically binds,” as used herein, is meant a molecule, such as an antibody, which recognizes and binds to another molecule or feature, but does not substantially recognize or bind other molecules or features in a sample.

As used herein, the term “transdominant negative mutant gene” refers to a gene encoding a polypeptide or protein product that prevents other copies of the same gene or gene product, which have not been mutated (i.e., which have the wild-type sequence) from functioning properly (e.g., by inhibiting wild type protein function). The product of a transdominant negative mutant gene is referred to herein as “dominant negative” or “DN” (e.g., a dominant negative protein, or a DN protein).

“Therapeutically effective amount” is an amount of a compound of the invention, that when administered to a patient, ameliorates a symptom of the disease. The amount of a compound of the invention which constitutes a “therapeutically effective amount” will vary depending on the compound, the disease state and its severity, the age of the patient to be treated, and the like. The therapeutically effective amount can be determined routinely by one of ordinary skill in the art having regard to his own knowledge and to this disclosure.

“Patient” for the purposes of the present invention includes humans and other animals, particularly mammals, and other organisms. Thus the methods are applicable to both human therapy and veterinary applications. In a preferred embodiment the patient is a mammal, and in a most preferred embodiment the patient is human.

The terms “treat,” “treating,” and “treatment,” refer to therapeutic or preventative measures described herein. The methods of “treatment” employ administration to a subject, in need of such treatment, a composition of the present invention, for example, a subject having a disorder mediated by ALK or other oncoprotein or a subject who ultimately may acquire such a disorder, in order to prevent, cure, delay, reduce the severity of, or ameliorate one or more symptoms of the disorder or recurring disorder, or in order to prolong the survival of a subject beyond that expected in the absence of such treatment.

The phrase “inhibit,” as used herein, means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down regulate a protein, a gene, and an mRNA stability, expression, function and activity, e.g., antagonists.

As used herein, a “marker gene” or “reporter gene” is a gene that imparts a distinct phenotype to cells expressing the gene and thus permits cells having the gene to be distinguished from cells that do not have the gene. Such genes may encode either a selectable or screenable marker, depending on whether the marker confers a trait which one can ‘select’ for by chemical means, i.e., through the use of a selective agent (e.g., a herbicide, antibiotic, or the like), or whether it is simply a “reporter” trait that one can identify through observation or testing, i.e., by ‘screening’. Elements of the present disclosure are exemplified in detail through the use of particular marker genes. Of course, many examples of suitable marker genes or reporter genes are known to the art and can be employed in the practice of the invention. Therefore, it will be understood that the following discussion is exemplary rather than exhaustive. In light of the techniques disclosed herein and the general recombinant techniques which are known in the art, the present invention renders possible the alteration of any gene.

Exemplary genes include, but are not limited to, a neo gene, a β-gal gene, a gus gene, a cat gene, a gpt gene, a hyg gene, a hisD gene, a ble gene, a mprt gene, a bar gene, a nitrilase gene, a mutant acetolactate synthase gene (ALS) or acetoacid synthase gene (AAS), a methotrexate-resistant dhfr gene, a dalapon dehalogenase gene, a mutated anthranilate synthase gene that confers resistance to 5-methyl tryptophan (WO 97/26366), an R-locus gene, a β-lactamase gene, a xy/E gene, an α-amylase gene, a tyrosinase gene, a luciferase (luc) gene, (e.g., a Renilla reniformis luciferase gene, a firefly luciferase gene, or a click beetle luciferase (Pyrophorus plagiophthalamus) gene, an aequorin gene, or a green fluorescent protein gene. Included within the terms selectable or screenable marker genes are also genes which encode a “secretable marker” whose secretion can be detected as a means of identifying or selecting for transformed cells. Examples include markers which encode a secretable antigen that can be identified by antibody interaction, or even secretable enzymes which can be detected by their catalytic activity. Secretable proteins fall into a number of classes, including small, diffusible proteins detectable, e.g., by ELISA, and proteins that are inserted or trapped in the cell membrane.

The term “transfected” or “transformed” or “transduced” as used herein refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

Inhibition of DLK palmitoylation is a neuroprotective strategy and DLK palmitoylation inhibitors represent a new class of neuroprotectants that could be either complementary or superior to existing therapeutic options.

The palmitoylation-dependence of DLK-mediated degeneration is exploited herein to identify novel neuroprotective compounds. In one aspect, the present invention provides a screening method using a cell-based assay of DLK palmitoylation. In one embodiment, the assay is used in a High-Content Imaging screen to identify small molecules or compounds that prevent DLK palmitoylation. In one embodiment, initial ‘hits’ are validated and then investigated with a secondary assay to determine whether such compounds can protect against neurodegeneration. In certain embodiments the compounds identified in the screen are classified and the functional groups are identified in order to identify a novel class of neuroprotectants.

Identified compounds can be used, for example, for the treatment or prevention of neurodegeneration following acute injury (stroke, traumatic brain injury, peripheral nerve injury); treatment or prevention of neurodegeneration in chronic conditions (Alzheimer's Disease, Amyotrophic Lateral Sclerosis); amelioration of symptoms for those suffering from chemotherapy-induced peripheral neuropathy (CIPN), diabetic neuropathy, HIV-associated neuropathies; treatments for conditions in which aberrant palmitoylation likely contributes to disease progression e.g. certain cancers; basic research into neurodegeneration mechanism; and basic research into roles of palmitoylation in other tissues and cells.

### Screening Assay

The present invention provides a system and method to screen and identify compounds that modulate palmitoylation of a protein, including palmitoylation of DLK. In one embodiment, the systems and methods comprise high content screening (HCS) of suitable compounds. In some instances, HCS is a screening method that uses live cells to perform a series of experiments as the basis for high throughput compound discovery. Typically, HCS is an automated system to enhance the throughput of the screening process. However, the present invention is not limited to the speed or automation of the screening process.

As described elsewhere herein, palmitoylation of DLK is associated with neurodegeneration. The present invention comprises an HCS assay to screen for compounds that modulate DLK palmitoylation. In one embodiment, the compounds are screened for the ability to inhibit DLK palmitoylation. Non-limiting examples of the mechanism of action for candidate compounds include, but is not limited to, broad spectrum palmitoylation inhibitors, inhibitors of specific palmitoyl acyltransferases (PATs), and allosteric modulators of DLK conformation that prevent is palmitoylation.

In one embodiment, the HCS assay of the invention provides for a system to generate high quality “hits” identifying compounds that modulate palmitoylation.

In another embodiment of the invention, the HCS assay provides for a high throughput assay. Preferably, the assay provides automated screening of thousands of test compounds. Compounds tested in the screening method of the present invention are not limited to the specific type of the compound. Non-limiting examples of potential test compounds include chemical agents, pharmaceuticals, small molecules, peptides, proteins (such as antibodies, cytokines, enzymes, etc.), and nucleic acids, including gene medicines and introduced genes, which may encode therapeutic agents such as proteins, antisense agents (i.e. nucleic acids comprising a sequence complementary to a target RNA expressed in a target cell type, such as RNAi or siRNA), ribozymes, etc. Additionally or alternatively, the assay of the invention may screen a physical agent such as radiation (e.g. ionizing radiation, UV-light or heat); these can be tested alone or in combination with chemical and other agents. In one embodiment, entire compound libraries are screened. Compound libraries are a large collection of stored compounds utilized for high throughput screening. Compounds in a compound library can have no relation to one another, or alternatively have a common characteristic. For example, a hypothetical compound library may contain all known compounds known to bind to a specific binding region. As would be understood by one skilled in the art, the methods of the invention are not limited to the types of compound libraries screened. Non-limiting examples of compound libraries include the sets from Prestwick, LOPAC, Chembridge, Maybridge, LifeChemicals and the NIH Clinical Collection.

In one embodiment, the assay of the invention may also be used to test delivery vehicles. These may be of any form, from conventional pharmaceutical formulations, to gene delivery vehicles. For example, the assay may be used to compare the effects of the same compound administered by two or more different delivery systems (e.g. a depot formulation and a controlled release formulation). It may also be used to investigate whether a particular vehicle could have effects of itself on palmitoylation. As the use of gene-based therapeutics increases, the safety issues associated with the various possible delivery systems become increasingly important. Thus the models of the present invention may be used to investigate the properties of delivery systems for nucleic acid therapeutics, such as naked DNA or RNA, viral vectors (e.g. retroviral or adenoviral vectors), liposomes, etc. Thus the test compound may be a delivery vehicle of any appropriate type with or without any associated therapeutic agent.

In one embodiment, compounds are evaluated alone. In another embodiment, compounds are evaluated when delivered along with a delivery vehicle. Non-limiting examples of delivery vehicles include polymersomes, vesicles, micelles, plasmid vectors, viral vectors, and the like. As described elsewhere herein, compounds are evaluated for their ability to modulate palmitoylation. In another embodiment, the methods of the invention comprise selecting a compound that modulates palmitoylation from a compound library. In another embodiment, test compounds are delivered along with known therapeutic agents to determine whether the test compounds exhibit interference or synergy with other agents.

The test compound may be added to the assay to be tested by any suitable means. For example, the test compound may be injected into the cells of the assay, or it can be added to the nutrient medium and allowed to diffuse into the cells. The assay is also suitable for testing the effects of physical agents such as ionizing radiation, UV-light or heat alone or in combination with chemical agents (for example, in photodynamic therapy).

In situations where “high-throughput” modalities are preferred, it is typical to that new chemical entities with useful properties are generated by identifying a chemical compound (called a “hit compound”) with some desirable property or activity, and evaluating the property of those compounds. A non-limiting example of a high-throughput screening assay is to array the membrane of the invention to 96, 384, 1536, etc. well or slot format to enable a full high throughput screen.

In one embodiment, high throughput screening methods involve providing a library containing a large number of compounds (candidate compounds) potentially having the desired activity. Such “combinatorial chemical libraries” are then screened in one or more assays, as described herein, to identify those library members (particular chemical species or subclasses) that display a desired characteristic activity. The compounds thus identified can serve as conventional “hit compounds” or can themselves be used as potential or actual therapeutics. As further discussed below, in one embodiment, the screen and method of the present invention comprise a primary screen, one or more counter screens, one or more orthogonal screens, or one or more secondary screens. In one embodiment, one or more of the primary screen, counter screens, orthogonal screens, and secondary screens is a high throughput screen or high content screen, as described elsewhere herein.

### Primary Screen

The system and methods of the invention is based upon the detection of the localization of DLK in a living cell or fixed cell. In one embodiment, the system and methods of the invention comprise a primary screen. In one embodiment, the primary screen comprises the acquisition of images of cells to detect DLK localization. In one embodiment, the primary screen comprises the acquisition of images of cells to detect membrane association of DLK. Localization or membrane association of DLK is made through the detection of a signal corresponding to DLK. In one embodiment, the screen of the invention comprises the use of cells that do not natively express DLK. In one embodiment, cells are genetically modified to express DLK. The present invention is not limited to cells expressing full-length DLK protein. One skilled in the art would appreciate the screen of the present invention can use cells which are modified to express only a specific region or regions of DLK, for example a fragment of DLK containing the palmitoylation site. In one embodiment, cells of the screen express DLK protein that is tagged with a detectable marker, for example fluorescently tagged DLK. Non-limiting examples of fluorescent tags include green fluorescent protein (GFP), cyan fluorescent protein (CFP), yellow fluorescent protein (YFP), red fluorescent protein (RFP), orange fluorescent protein (OFP), eGFP, mCherry, hrGFP, hrGFPII, and the like. Fluorescent tags may also be photoconvertable such as for example kindling red fluorescent protein (KFP-red), PS-CFP2, Dendra2, CoralHue Kaede and CoralHue Kikume. However, the invention should not be limited to a particular label. Rather, any detectable label can be used to tag DLK.

In one embodiment, the screen comprises a cell or cell population modified to express DLK and/or other proteins of interest. In one embodiment, the cell or cell population is modified by administering an expression vector encoding the protein of interest. As would be understood by those skilled in the art, the expression vector used to modify the cell or cell population of the screen includes any vector known in the art such as cosmids, plasmids, phagemid, lentiviral vectors, adenoviral vectors, retroviral vectors, adeno-associated vectors, and the like.

The expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used. In one embodiment, the cell or cell population of the screen are administered a lentiviral vector encoding DLK.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

Employing genetic engineering technology necessarily requires growing recombinant host cells (e.g., transfectants, transformants) under a variety of specified conditions as determined by the requirements of the cells and the particular cellular state desired by the practitioner. In one embodiment, genetic engineering includes transiently transfected cells or the establishment of stable expression cell lines. For example, a host cell may possess (as determined by its genetic disposition) certain nutritional requirements, or a particular resistance or sensitivity to physical (e.g., temperature) and/or chemical (e.g., antibiotic) conditions. In addition, specific culture conditions may be necessary to regulate the expression of a desired gene (e.g., the use of inducible promoters), or to initiate a particular cell state (e.g., yeast cell mating or sporulation). These varied conditions and the requirements to satisfy such conditions are understood and appreciated by practitioners in the art.

The recombinant vectors harboring the sequence encoding DLK, or other elements of the present invention, can be introduced into an appropriate host cell by any means known in the art. For example, the vector can be transfected into the host cell by calcium phosphate co-precipitation, by conventional mechanical procedures such as microinjection or electroporation, by insertion of a plasmid encased in liposomes, and by virus vectors. These techniques are all well-known and routinely practiced in the art, e.g., Brent et al., Current Protocols in Molecular Biology, John Wiley & Sons, Inc. (ringbou ed., 2003); and Weissbach & Weissbach, Methods for Plant Molecular Biology, Academic Press, NY, Section VIII, pp. 42 1-463, 1988. Host cells which harbor the transfected recombinant vector can be identified and isolated using the selection marker present on the vector. Large numbers of recipient cells may then be grown in a medium which selects for vector-containing cells. These cells may be used directly or the expressed recombinant protein may be purified in accordance with conventional methods such as extraction, precipitation, chromatography, affinity methods, electrophoresis and the like. The exact procedure used will depend upon the specific protein produced and the specific vector/host expression system utilized.

In an embodiment, host cells for expressing the recombinant vectors are eukaryotic cells. Eukaryotic vector/host systems, and mammalian expression systems, allow for proper post-translational modifications of expressed mammalian proteins to occur, e.g., proper processing of the primary transcript, glycosylation, phosphorylation and advantageously secretion of expressed product. Therefore, eukaryotic cells such as mammalian cells can be the host cells for the protein of a polypeptide of interest. Examples of such host cell lines include CHO, BHK, HEK293, VERO, HeLa, COS, MDCK, NS0 and W138. Such cells lines can be transiently transfected with DLK and/or other elements of the invention. Alternatively, stable cell lines genetically altered to constitutively express DLK and/or other elements of the invention can be generated by methods known in the art.

In some embodiments, engineered mammalian cell systems that utilize recombinant viruses or viral elements to direct expression of the protein of interest are employed. For example, when using adenovirus expression vectors, the coding sequence of DLK or other protein of interest may be ligated to an adenovirus transcription/translation control complex, e.g., the late promoter and tripartite leader sequence. This chimeric gene may then be inserted into the adenovirus genome by in vitro or in vivo recombination. Insertion in a non-essential region of the viral genome (e.g., region E1 or E3) will result in a recombinant virus that is viable and capable of expressing the polypeptide of interest in infected hosts (e.g., see Logan & Shenk, 1984 Proc. Natl. Acad. Sci. USA 81:3655-3659). Alternatively, the vaccinia virus 7.5K promoter may be used. (e.g., see, Mackett et al., 1982, Proc. Natl. Acad. Sci. USA, 79:7415-7419; Mackett et al., 1984, J. Virol. 49:857-864; Panicali et al., 1982, Proc. Natl. Acad. Sci. USA, 79:4927-4931). In certain embodiments, vectors are based on bovine papilloma virus which has the ability to replicate as extrachromasomal elements (Sarver et al., 1981, Mol. Cell. Biol. 1:486). These vectors can be used for stable expression by including a selectable marker in the plasmid, such as the neo gene. Alternatively, the retroviral genome can be modified for use as a vector capable of introducing and directing the expression of the gene of interest in host cells (Cone & Mulligan, 1984, Proc. Natl. Acad. Sci. USA 8 1:6349-6353). High level expression may also be achieved using inducible promoters, including, but not limited to, the metallothionine IIA promoter and heat shock promoters.

In one embodiment, the cells of the screen are modified to transiently express DLK. In another embodiment, the cells of the screen are modified for the stable expression of DLK. For example, in one embodiment, a cell line which stably expresses DLK, is generated and maintained under standard culturing protocols known in the art. In one embodiment, a cell of the screen comprises a nucleic acid encoding DLK

The present invention is related to screening methods comprising the automated detection of the cellular localization of proteins. In one embodiment, the localization of DLK or membrane association of DLK, or other elements of interest, is determined from images taken of cells expressing DLK, or other elements of interest. The localization of DLK or membrane association of DLK, may be determined in the live cell of the assay, or alternatively after the cell has been fixed. The present invention is not limited to the type or mode of microscopy utilized in imaging of the cells of the screen. In one embodiment, acquired images obtained through standard fluorescent microscopy techniques known in the art, detects the localization of the fluorescent signal in a cell, thereby detecting the localization of DLK within a cell. Under control conditions, palmitoylated DLK (palmityol-DLK) is associated with intracellular membranes in the cell, while inhibition of DLK palmitoylation results in the disruption of the membrane-association of DLK.

Thus, in embodiments wherein cells express fluorescently tagged DLK, images of the cells exhibit localization of fluorescence to intracellular membranes, which in certain embodiments, can be detected by detecting fluorescent puncta. In embodiments, where palmitoylation of DLK is inhibited, fluorescence would be observed to be more diffuse through the cell, which in certain embodiments, can be detected by detecting a decrease or lack of fluorescent puncta, as compared to control conditions where palmitoylation of DLK is not inhibited.

As would be understood by those skilled in the art, full length DLK protein, or portions thereof, can be used in the screening methods of the invention. For example, in one embodiment, cells of the screen comprise full length DLK In another embodiment, cells of the invention comprise only specific regions of DLK known to influence palmitoylation.

In one embodiment, the primary screen of the invention comprises the step of adding a compound known to inhibit palmitoylation, to be used, for example, as a positive control. An exemplary compound that inhibits DLK palmitoylation, and that can be used in the methods of the invention, is 2-Br.

In one embodiment, localization of DLK is quantitatively determined by the automated calculation of the DLK puncta. In control conditions, untreated cells (or vehicle treated) have a relative high number of DLK puncta, indicating membrane association of DLK. Cells treated with 2-Br would have a relative low number of DLK puncta. Test compounds that inhibit palmitoylation, and therefore designated as hits would be identified as having a relative low number of DLK puncta. In one embodiment, hits are defined as those test compounds that inhibit DLK membrane association by greater than 90%. In another embodiment, hits are defined as those test compounds that inhibit DLK membrane association by greater than 80%. In another embodiment, hits are defined as those test compounds that inhibit DLK membrane association by greater than 70%. In another embodiment, hits are defined as those test compounds that inhibit DLK membrane association greater than 60%. In another embodiment, hits are defined as those test compounds that inhibit DLK membrane association by greater than 50%. In another embodiment, hits are defined as those test compounds that inhibit DLK membrane association by greater than 40%. In another embodiment, hits are defined as those test compounds that inhibit DLK membrane association by greater than 30%. In another embodiment, hits are defined as those test compounds that inhibit DLK membrane association by greater than 20%. In another embodiment, hits are defined as those test compounds that inhibit DLK membrane association by greater than 10%.

The systems and methods of the invention comprise the steps of delivering a test compound to a cell expressing DLK and observing the localization of DLK or membrane association of DLK in response to the compound.

HCS assays typically comprise automated screening techniques to generate a high level of information from an experiment. In one embodiment, the system of the invention comprises numerous test compounds screened on cells cultured on a multi-well plate. Non-limiting examples of multi-well plates include a 6-well plate, a 24-well plate, a 96-well plate, and a 384-well plate. As such, each well comprises its own individual experiment detecting the response to a single test compound. In one embodiment, LMB treated alone and untreated or vehicle treated negative controls are conducted on each multi-well plate. Statistical analysis performed on the control wells enable the determination of the overall quality of experimentation done on the entire plate. In plates with controls determined to pass a statistical standard, test compounds that reduce DLK membrane association by a pre-defined amount relative to the mean of all compounds tested on the plate, that are not acutely cytotoxic and/or fluorescent outliers are flagged as “hits” as modulators of DLK palmitoylation. As such, the primary screen of the invention narrows a first population of test compounds into a second, smaller, population of test compounds that retain the ability to modulate DLK palmitoylation.

### Secondary Assays

The present invention is directed towards methods of identifying modulators of DLK palmitoylation. In one aspect of the invention, the methods comprise secondary assays of compounds identified in the primary screens of the invention, described elsewhere herein. Such secondary assays include, but is not limited to, evaluation of the cytotoxicity of identified compounds, investigation of the potential of identified compounds to modulate neurodegeneration, determination of the potency of identified compounds, and the determination of the mechanism of action of identified compounds.

### Composition

As described elsewhere herein, the invention provides a modulator (e.g., an inhibitor or activator) of palmitoylation. In various embodiment, the present invention includes compositions for modulating the level or activity of DLK palmitoylation.

In an embodiment of the present invention, the composition inhibits DLK palmitoylation. In one embodiment, the composition inhibits the specific palmitoyl acyltransferases (PATs) that palmitoylate DLK. In one embodiment, the composition can comprise an antibody or a fragment thereof, a peptide, a nucleic acid, or a small molecule.

The composition of the invention includes compositions for treating or preventing a disease or disorder associated with the activity of a palmitoylated protein, including, but not limited to, palmitoylated DLK. In one embodiment, the disease or disorder is a neurological disease or disorder.

The present invention also provides a compound selected from a pool of inhibitors for DLK, where the pool is identified by screening methods of the invention. In one exemplary embodiment, the compound identified through screening methods is Ketoconazole, including but is not limited to derivatives and analogs thereof.

Small Molecule

A small molecule may be obtained using standard methods known to the skilled artisan. Such methods include chemical organic synthesis or biological means. Biological means include purification from a biological source, recombinant synthesis and in vitro translation systems, using methods well known in the art. In one embodiment, a small molecule activator of the invention comprises an organic molecule, inorganic molecule, biomolecule, synthetic molecule, and the like.

Combinatorial libraries of molecularly diverse chemical compounds potentially useful in treating a variety of diseases and conditions are well known in the art as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores.

The small molecule and small molecule compounds described herein may be present as salts even if salts are not depicted and it is understood that the invention embraces all salts and solvates of the compounds depicted here, as well as the non-salt and non-solvate form of the compounds, as is well understood by the skilled artisan. In some embodiments, the salts of the compounds of the invention are pharmaceutically acceptable salts.

Where tautomeric forms may be present for any of the compounds described herein, each and every tautomeric form is intended to be included in the present invention, even though only one or some of the tautomeric forms may be explicitly depicted. For example, when a 2-hydroxypyridyl moiety is depicted, the corresponding 2-pyridone tautomer is also intended.

The invention also includes any or all of the stereochemical forms, including any enantiomeric or diasteriomeric forms of the compounds described. The recitation of the structure or name herein is intended to embrace all possible stereoisomers of compounds depicted. All forms of the compounds are also embraced by the invention, such as crystalline or non-crystalline forms of the compounds. Compositions comprising a compound of the invention are also intended, such as a composition of substantially pure compound, including a specific stereochemical form thereof, or a composition comprising mixtures of compounds of the invention in any ratio, including two or more stereochemical forms, such as in a racemic or non-racemic mixture.

In one embodiment, the small molecule compound of the invention comprises an analog or derivative of a compound described herein.

In one embodiment, the small molecules described herein are candidates for derivatization. As such, in certain instances, the analogs of the small molecules described herein that have modulated potency, selectivity, and solubility are included herein and provide useful leads for drug discovery and drug development. Thus, in certain instances, during optimization new analogs are designed considering issues of drug delivery, metabolism, novelty, and safety.

In some instances, small molecule activators described herein are derivatized/analoged as is well known in the art of combinatorial and medicinal chemistry. The analogs or derivatives can be prepared by adding and/or substituting functional groups at various locations. As such, the small molecules described herein can be converted into derivatives/analogs using well known chemical synthesis procedures. For example, all of the hydrogen atoms or substituents can be selectively modified to generate new analogs. Also, the linking atoms or groups can be modified into longer or shorter linkers with carbon backbones or hetero atoms. Also, the ring groups can be changed so as to have a different number of atoms in the ring and/or to include hetero atoms. Moreover, aromatics can be converted to cyclic rings, and vice versa. For example, the rings may be from 5-7 atoms, and may be homocycles or heterocycles.

As used herein, the term “analog”, “analogue,” or “derivative” is meant to refer to a chemical compound or molecule made from a parent compound or molecule by one or more chemical reactions. As such, an analog can be a structure having a structure similar to that of the small molecule compounds described herein or can be based on a scaffold of a small molecule compound described herein, but differing from it in respect to certain components or structural makeup, which may have a similar or opposite action metabolically. An analog or derivative of any of a small molecule compound in accordance with the present invention can be used to inhibit DLK palmitoylation, including but is not limited to inhibiting the specific palmitoyl acyltransferases (PATs) that palmitoylate DLK.

In one embodiment, the small molecule compounds described herein can independently be derivatized/analoged by modifying hydrogen groups independently from each other into other substituents. That is, each atom on each molecule can be independently modified with respect to the other atoms on the same molecule. Any traditional modification for producing a derivative/analog can be used. For example, the atoms and substituents can be independently comprised of hydrogen, an alkyl, aliphatic, straight chain aliphatic, aliphatic having a chain hetero atom, branched aliphatic, substituted aliphatic, cyclic aliphatic, heterocyclic aliphatic having one or more hetero atoms, aromatic, heteroaromatic, polyaromatic, polyamino acids, peptides, polypeptides, combinations thereof, halogens, halo-substituted aliphatics, and the like. Additionally, any ring group on a compound can be derivatized to increase and/or decrease ring size as well as change the backbone atoms to carbon atoms or hetero atoms.

Nucleic Acids

In certain embodiments, the composition comprises a modulator of palmitoylation including but is not limited to DLK palmitoylation and palmitoyl acyltransferases (PATs) that palmitoylate DLK.

In other related aspects, the invention includes an isolated nucleic acid. In some instances, the modulator is an siRNA, antisense molecule, or CRISPR guide RNA, which inhibits one or more of DLK palmitoylation and palmitoyl acyltransferases (PATs) that palmitoylate DLK. In one embodiment, the nucleic acid comprises a promoter/regulatory sequence such that the nucleic acid is preferably capable of directing expression of the nucleic acid. Thus, the invention encompasses expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York), and in Ausubel et al. (2008, Current Protocols in Molecular Biology, John Wiley & Sons, New York) and as described elsewhere herein. In one embodiment, siRNA is used to inhibit one or more of DLK palmitoylation and palmitoyl acyltransferases (PATs) that palmitoylate DLK. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216.

In another aspect, the invention includes a vector comprising an siRNA or antisense polynucleotide. In one embodiment, the siRNA or antisense polynucleotide is capable of inhibiting the expression of a target polypeptide. In one embodiment, the siRNA or antisense polynucleotide is capable of decreasing the expression of a target miRNA. The incorporation of a desired polynucleotide into a vector and the choice of vectors is well-known in the art as described in, for example, Sambrook et al., supra, and Ausubel et al., supra, and elsewhere herein.

Following the generation of the polynucleotide, a skilled artisan will understand that the polynucleotide will have certain characteristics that can be modified to improve the polynucleotide as a therapeutic compound. Therefore, the polynucleotide may be further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrwal et al., 1987 Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queosine, and wybutosine and the like, as well as acetyl- methyl-, thio- and other modified forms of adenine, cytidine, guanine, thymine, and uridine.

Polypeptides

In other related aspects, the invention includes an isolated peptide that inhibits one or more of DLK palmitoylation and palmitoyl acyltransferases (PATs) that palmitoylate DLK. For example, in one embodiment, the peptide of the invention can binding to, competing with, or acting as a transdominant negative mutant of a protein which activates one or more of DLK palmitoylation and palmitoyl acyltransferases (PATs) that palmitoylate DLK, thereby inhibiting the activation of DLK palmitoylation.

The variants of the polypeptides according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

The polypeptides of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The polypeptides of the invention may include unnatural amino acids formed by post-translational modification or by introducing unnatural amino acids during translation. A variety of approaches are available for introducing unnatural amino acids during protein translation. By way of example, special tRNAs, such as tRNAs which have suppressor properties, suppressor tRNAs, have been used in the process of site-directed non-native amino acid replacement (SNAAR). In SNAAR, a unique codon is required on the mRNA and the suppressor tRNA, acting to target a non-native amino acid to a unique site during the protein synthesis (described in WO90/05785). However, the suppressor tRNA must not be recognizable by the aminoacyl tRNA synthetases present in the protein translation system. In certain cases, a non-native amino acid can be formed after the tRNA molecule is aminoacylated using chemical reactions which specifically modify the native amino acid and do not significantly alter the functional activity of the aminoacylated tRNA. These reactions are referred to as post-aminoacylation modifications. For example, the epsilon-amino group of the lysine linked to its cognate tRNA (tRNA_{LYS}), could be modified with an amine specific photoaffinity label.

A peptide of the invention may be conjugated with other molecules, such as proteins, to prepare fusion proteins. This may be accomplished, for example, by the synthesis of N-terminal or C-terminal fusion proteins provided that the resulting fusion protein retains the functionality of the peptide.

Cyclic derivatives of the peptides or chimeric proteins of the invention are also part of the present invention. Cyclization may allow the peptide or chimeric protein to assume a more favorable conformation for association with other molecules. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid as described by Ulysse, L., et al., J. Am. Chem. Soc. 1995, 117, 8466-8467. The components that form the bonds may be side chains of amino acids, non-amino acid components or a combination of the two. In an embodiment of the invention, cyclic peptides may comprise a beta-turn in the right position. Beta-turns may be introduced into the peptides of the invention by adding the amino acids Pro-Gly at the right position.

In other embodiments, the subject peptide therapeutics are peptidomimetics of the peptides. Peptidomimetics are compounds based on, or derived from, peptides and proteins. The peptidomimetics of the present invention typically can be obtained by structural modification of a known peptide sequence using unnatural amino acids, conformational restraints, isosteric replacement, and the like. The subject peptidomimetics constitute the continuum of structural space between peptides and non-peptide synthetic structures; peptidomimetics may be useful, therefore, in delineating pharmacophores and in helping to translate peptides into nonpeptide compounds with the activity of the parent peptides.

Moreover, as is apparent from the present disclosure, mimetopes of the subject peptide can be provided. Such peptidomimetics can have such attributes as being non-hydrolyzable (e.g., increased stability against proteases or other physiological conditions which degrade the corresponding peptide), increased specificity and/or potency, and increased cell permeability for intracellular localization of the peptidomimetic.

Peptides of the invention may be developed using a biological expression system. The use of these systems allows the production of large libraries of random peptide sequences and the screening of these libraries for peptide sequences that bind to particular proteins. Libraries may be produced by cloning synthetic DNA that encodes random peptide sequences into appropriate expression vectors. (see Christian et al 1992, J. Mol. Biol. 227:711; Devlin et al, 1990 Science 249:404; Cwirla et al 1990, Proc. Natl. Acad, Sci. USA, 87:6378). Libraries may also be constructed by concurrent synthesis of overlapping peptides (see U.S. Pat. No. 4,708,871).

The peptides and chimeric proteins of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

Antibodies and peptides may be modified using ordinary molecular biological techniques to improve their resistance to proteolytic degradation or to optimize solubility properties or to render them more suitable as a therapeutic agent. Analogs of such polypeptides include those containing residues other than naturally occurring L-amino acids, e.g., D-amino acids or non-naturally occurring synthetic amino acids. The polypeptides useful in the invention may further be conjugated to non-amino acid moieties that are useful in their application. In particular, moieties that improve the stability, biological half-life, water solubility, and immunologic characteristics of the peptide are useful. A non-limiting example of such a moiety is polyethylene glycol (PEG).

Antibodies

In one embodiment, the invention includes an antibody, or antibody fragment, specific to DLK to inhibit DLK palmitoylation. In another embodiment, the antibody or fragment thereof inhibits palmitoyl acyltransferases (PATs) that palmitoylate DLK.

The invention also contemplates an antibody, or antibody fragment, specific for a protein which activates one or more of DLK palmitoylation and palmitoyl acyltransferases (PATs) that palmitoylate DLK.

Methods of making and using antibodies are well known in the art. For example, polyclonal antibodies useful in the present invention are generated by immunizing rabbits according to standard immunological techniques well-known in the art (see, e.g., Greenfield et al., 2014, Antibodies, A Laboratory Manual, Cold Spring Harbor, N.Y.). Such techniques include immunizing an animal with a chimeric protein comprising a portion of another protein such as a maltose binding protein or glutathione (GSH) tag polypeptide portion, and/or a moiety such that the antigenic protein of interest is rendered immunogenic (e.g., an antigen of interest conjugated with keyhole limpet hemocyanin, KLH) and a portion comprising the respective antigenic protein amino acid residues. The chimeric proteins are produced by cloning the appropriate nucleic acids encoding the marker protein into a plasmid vector suitable for this purpose, such as but not limited to, pMAL-2 or pCMX.

One skilled in the art would appreciate, based upon the disclosure provided herein, that the antibody can specifically bind with any portion of the antigen and the full-length protein can be used to generate antibodies specific therefor. However, the present invention is not limited to using the full-length protein as an immunogen. Rather, the present invention includes using an immunogenic portion of the protein to produce an antibody that specifically binds with a specific antigen. That is, the invention includes immunizing an animal using an immunogenic portion, or antigenic determinant, of the antigen.

Once armed with the sequence of a specific antigen of interest and the detailed analysis localizing the various conserved and non-conserved domains of the protein, the skilled artisan would understand, based upon the disclosure provided herein, how to obtain antibodies specific for the various portions of the antigen using methods well-known in the art or to be developed.

The skilled artisan would appreciate, based upon the disclosure provided herein, that that present invention includes use of a single antibody recognizing a single antigenic epitope but that the invention is not limited to use of a single antibody. Instead, the invention encompasses use of at least one antibody where the antibodies can be directed to the same or different antigenic protein epitopes.

The generation of polyclonal antibodies is accomplished by inoculating the desired animal with the antigen and isolating antibodies which specifically bind the antigen therefrom using standard antibody production methods.

Monoclonal antibodies directed against full length or peptide fragments of a protein or peptide may be prepared using any well-known monoclonal antibody preparation procedures. Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art. Further, the antibody of the invention may be “humanized” using methods of humanizing antibodies well-known in the art or to be developed.

The present invention also includes the use of humanized antibodies specifically reactive with epitopes of an antigen of interest. The humanized antibodies of the invention have a human framework and have one or more complementarity determining regions (CDRs) from an antibody, typically a mouse antibody, specifically reactive with an antigen of interest.

The invention also includes functional equivalents of the antibodies described herein. Functional equivalents have binding characteristics comparable to those of the antibodies, and include, for example, hybridized and single chain antibodies, as well as fragments thereof.

Functional equivalents include polypeptides with amino acid sequences substantially the same as the amino acid sequence of the variable or hypervariable regions of the antibodies. “Substantially the same” amino acid sequence is defined herein as a sequence with at least 70%, preferably at least about 80%, more preferably at least about 90%, even more preferably at least about 95%, and most preferably at least 99% homology to another amino acid sequence (or any integer in between 70 and 99), as determined by the FASTA search method. Chimeric or other hybrid antibodies have constant regions derived substantially or exclusively from human antibody constant regions and variable regions derived substantially or exclusively from the sequence of the variable region of a monoclonal antibody from each stable hybridoma.

Single chain antibodies (scFv) or Fv fragments are polypeptides that consist of the variable region of the heavy chain of the antibody linked to the variable region of the light chain, with or without an interconnecting linker. Thus, the Fv comprises an antibody combining site.

Functional equivalents of the antibodies of the invention further include fragments of antibodies that have the same, or substantially the same, binding characteristics to those of the whole antibody. Such fragments may contain one or both Fab fragments or the F(ab′)₂ fragment. The antibody fragments contain all six complement determining regions of the whole antibody, although fragments containing fewer than all of such regions, such as three, four or five complement determining regions, are also functional. The functional equivalents are members of the IgG immunoglobulin class and subclasses thereof, but may be or may combine with any one of the following immunoglobulin classes: IgM, IgA, IgD, or IgE, and subclasses thereof. Heavy chains of various subclasses, such as the IgG subclasses, are responsible for different effector functions and thus, by choosing the desired heavy chain constant region, hybrid antibodies with desired effector function are produced. Exemplary constant regions are gamma 1 (IgG1), gamma 2 (IgG2), gamma 3 (IgG3), and gamma 4 (IgG4). The light chain constant region can be of the kappa or lambda type.

The immunoglobulins of the present invention can be monovalent, divalent or polyvalent. Monovalent immunoglobulins are dimers (HL) formed of a hybrid heavy chain associated through disulfide bridges with a hybrid light chain. Divalent immunoglobulins are tetramers (H₂L₂) formed of two dimers associated through at least one disulfide bridge.

Uses of Identified Compounds

Modulators of palmitoylation that are identified by the systems and methods of the invention can be used in a variety of research and clinical methods. For example, the identified compounds can be used to study palmitoylation broadly, or palmitoylation of specific proteins specifically, in a variety of research settings.

In certain embodiments, the identified compounds can be used in the treatment or prevention of any disease or disorder association with palmitoylation. In one embodiment, the identified compounds can be used in the treatment or prevention of any disease or disorder associated with palmitoyl-DLK. For example, identified compounds can be used, for example, for the treatment or prevention of neurodegeneration following acute injury (stroke, traumatic brain injury, peripheral nerve injury); treatment or prevention of neurodegeneration in chronic conditions (Alzheimer's Disease, Amyotrophic Lateral Sclerosis); amelioration of symptoms for those suffering from chemotherapy-induced peripheral neuropathy (CIPN), diabetic neuropathy, and HIV-associated neuropathies.

Modulators identified through the methods of the invention, can be administered to a subject or patient through any means known in the art. Administration of the therapeutic agent in accordance with the present invention may be continuous or intermittent, depending, for example, upon the recipient's physiological condition, whether the purpose of the administration is therapeutic or prophylactic, and other factors known to skilled practitioners. The administration of the agents of the invention may be essentially continuous over a preselected period of time or may be in a series of spaced doses. Both local and systemic administration is contemplated. The amount administered will vary depending on various factors including, but not limited to, the composition chosen, the particular disease, the weight, the physical condition, and the age of the mammal, and whether prevention or treatment is to be achieved. Such factors can be readily determined by the clinician employing animal models or other test systems which are well known to the art

As contemplated elsewhere herein, inhibitors identified through methods of the invention may comprise nucleic acids, including DNA and RNA sequences. Pharmaceutical formulations, dosages and routes of administration for nucleic acids are generally disclosed, for example, in Felgner et al., 1987. Further, administration of proteins, peptides, siRNA and other compositions that display therapeutic benefit may be accomplished through administration of nucleic acid molecules that encode for such compositions (see, for example, Felgner et al. 1987, U.S. Pat. No. 5,580,859, Pardoll et al. 1995; Stevenson et al. 1995; Molling 1997; Donnelly et al. 1995; Yang et al. II; Abdallah et al. 1995)

One or more suitable unit dosage forms having the therapeutic agent(s) of the invention, which, as discussed below, may optionally be formulated for sustained release (for example using microencapsulation, see WO 94/07529, and U.S. Pat. No. 4,962,091 the disclosures of which are incorporated by reference herein), can be administered by a variety of routes including parenteral, including by intravenous and intramuscular routes, as well as by direct injection into the diseased tissue. For example, the therapeutic agent may be directly injected into the tumor. The formulations may, where appropriate, be conveniently presented in discrete unit dosage forms and may be prepared by any of the methods well known to pharmacy. Such methods may include the step of bringing into association the therapeutic agent with liquid carriers, solid matrices, semi-solid carriers, finely divided solid carriers or combinations thereof, and then, if necessary, introducing or shaping the product into the desired delivery system.

When the therapeutic agents of the invention are prepared for administration, they are preferably combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. The active ingredient for administration may be present as a powder or as granules; as a solution, a suspension or an emulsion.

Pharmaceutical formulations containing the therapeutic agents of the invention can be prepared by procedures known in the art using well known and readily available ingredients. The therapeutic agents of the invention can also be formulated as solutions appropriate for parenteral administration, for instance by intramuscular, subcutaneous or intravenous routes.

The pharmaceutical formulations of the therapeutic agents of the invention can also take the form of an aqueous or anhydrous solution or dispersion, or alternatively the form of an emulsion or suspension.

Thus, the therapeutic agent may be formulated for parenteral administration (e.g., by injection, for example, bolus injection or continuous infusion) and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in powder form, obtained by aseptic isolation of sterile solid or by lyophilization from solution, for constitution with a suitable vehicle, e.g., sterile, pyrogen-free water, before use.

It will be appreciated that the unit content of active ingredient or ingredients contained in an individual aerosol dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions, such as phosphate buffered saline solutions pH 7.0-8.0.

The expression vectors, transduced cells, polynucleotides and polypeptides (active ingredients) of this invention can be formulated and administered to treat a variety of disease states by any means that produces contact of the active ingredient with the agent's site of action in the body of the organism. They can be administered by any conventional means available for use in conjunction with pharmaceuticals, either as individual therapeutic active ingredients or in a combination of therapeutic active ingredients. They can be administered alone, but are generally administered with a pharmaceutical carrier selected on the basis of the chosen route of administration and standard pharmaceutical practice.

In general, water, suitable oil, saline, aqueous dextrose (glucose), and related sugar solutions and glycols such as propylene glycol or polyethylene glycols are suitable carriers for parenteral solutions. Solutions for parenteral administration contain the active ingredient, suitable stabilizing agents and, if necessary, buffer substances. Antioxidizing agents such as sodium bisulfate, sodium sulfite or ascorbic acid, either alone or combined, are suitable stabilizing agents. Also used are citric acid and its salts and sodium Ethylenediaminetetraacetic acid (EDTA). In addition, parenteral solutions can contain preservatives such as benzalkonium chloride, methyl- or propyl-paraben and chlorobutanol. Suitable pharmaceutical carriers are described in Remington's Pharmaceutical Sciences, a standard reference text in this field.

The active ingredients of the invention may be formulated to be suspended in a pharmaceutically acceptable composition suitable for use in mammals and in particular, in humans.

Additionally, standard pharmaceutical methods can be employed to control the duration of action. These are well known in the art and include control release preparations and can include appropriate macromolecules, for example polymers, polyesters, polyamino acids, polyvinyl, pyrolidone, ethylenevinylacetate, methyl cellulose, carboxymethyl cellulose or protamine sulfate. The concentration of macromolecules as well as the methods of incorporation can be adjusted in order to control release. Additionally, the agent can be incorporated into particles of polymeric materials such as polyesters, polyamino acids, hydrogels, poly (lactic acid) or ethylenevinylacetate copolymers. In addition to being incorporated, these agents can also be used to trap the compound in microcapsules.

Accordingly, the pharmaceutical composition of the present invention may be delivered via various routes and to various sites in a mammal body to achieve a particular effect. One skilled in the art will recognize that although more than one route can be used for administration, a particular route can provide a more immediate and more effective reaction than another route. Local or systemic delivery can be accomplished by administration comprising application or instillation of the formulation into body cavities, inhalation or insufflation of an aerosol, or by parenteral introduction, comprising intramuscular, intravenous, peritoneal, subcutaneous, intradermal, as well as topical administration.

The active ingredients of the present invention can be provided in unit dosage form wherein each dosage unit, e.g., a teaspoonful, tablet, solution, or suppository, contains a predetermined amount of the composition, alone or in appropriate combination with other active agents. The term “unit dosage form” as used herein refers to physically discrete units suitable as unitary dosages for human and mammal subjects, each unit containing a predetermined quantity of the compositions of the present invention, alone or in combination with other active agents, calculated in an amount sufficient to produce the desired effect, in association with a pharmaceutically acceptable diluent, carrier, or vehicle, where appropriate. The specifications for the unit dosage forms of the present invention depend on the particular effect to be achieved and the particular pharmacodynamics associated with the pharmaceutical composition in the particular host.

These methods described herein are by no means all-inclusive, and further methods to suit the specific application will be apparent to the ordinary skilled artisan. Moreover, the effective amount of the compositions can be further approximated through analogy to compounds known to exert the desired effect.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: Screening for Modulators of DLK Palmitoylation

Damage to the central nervous system (CNS) or peripheral nervous system (PNS), caused by trauma or disease, can lead to permanent neurological disability. Patients frequently suffer permanent loss of sensory and/or motor function, often accompanied by chronic pain. Traumatic brain injury (TBI) and pediatric stroke, are linked to CNS damage, while pediatric brachial plexus injury, is linked to PNS damage. Each of these conditions can greatly impair nervous system function, but each also presents opportunities for therapeutic intervention.

Much of the tissue damage following TBI or pediatric stroke is secondary to the initial trauma (Park, 2008; Manara, 2013). In brachial plexus injury, nerves that convey signals between the spine and the shoulder, arm and hand are damaged, leading to loss of sensory and/or motor function (Mannan, 2006). A clear strategy in each of these conditions is thus to reduce the impact of the initial trauma by limiting the size of the affected area, or the extent of neurodegeneration within it. Proteins that control neurodegeneration are promising therapeutic targets in this respect.

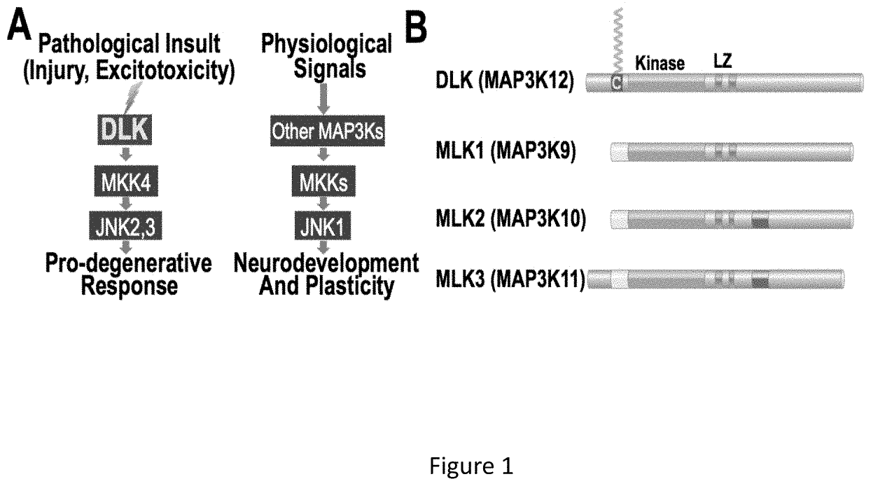

One group of proteins that is heavily implicated in neurodegeneration is the c-Jun N-terminal kinase (JNK) (Yang, 1997) family of Mitogen-activated protein kinases (MAPKs) (Pearson, 2001). Of the three mammalian JNK genes (JNK1-3), JNK2 and JNK3 are particularly critical for several forms of neurodegeneration (FIG. 1); JNK3 knockout mice are protected from neuronal death following excitotoxic insult (Yang, 1997; Kuan, 2003) or sciatic nerve axotomy (Keramaris, 2005), while JNK2/3 double knockouts show markedly reduced Retinal Ganglion Cell (RGC) death following optic nerve injury (Fernandes, 2012).

These findings led to the development of numerous JNK inhibitors to combat neurodegeneration (Siddiqui, 2010), yet these inhibitors appear only rarely effective in vivo (Carboni, 2008; Kamenecka, 2010). Some of this lack of success is likely due to solubility and/or blood brain barrier permeability issues, both of which are critical for CNS therapeutic efficacy (Kamenecka, 2010). However, few JNK inhibitors distinguish between JNK2/3 and the closely related JNK1 (Siddiqui, 2010), a key regulator of neuronal development, neuronal plasticity and higher brain function (Bjorkblom, 2005). Consistent with this notion, panJNK inhibitors actually worsen outcomes in multiple models of neurodegeneration (Wityak, 2015; Murata, 2012).

A more promising approach might thus be to identify specific ‘upstream’ enzymes that mediate pathological JNK2/3 activation but which are not involved in physiologic JNK1 activation. In this regard, there has been intense interest in Dual Leucine-zipper Kinase (DLK), a neural-specific ‘MAP3K’ that transduces pro-degenerative JNK signals without affecting basal physiological JNK activity (Ghosh, 2011, Hirai, 2005), FIG. 1). Indeed, knockout of DLK strikingly protects neurons from several forms of neurodegeneration (Ghosh, 2011; Pozniak, 2013). These findings spurred efforts to develop inhibitors of DLK's kinase activity (Welsbie, 2013), but it is troubling that the most promising DLK inhibitors reported thus far also inhibit homologous MAP3Ks and/or other kinases (Welsbie, 2013; Patel, 2015; Yin, 2016).

The lack of selectivity of DLK inhibitors is partly explained by the high degree of similarity between the kinase domains of DLK and its closest orthologs, the Mixed Lineage Kinase (MLK) family of MAP3Ks (FIG. 1B). Indeed, all three mammalian MLKs can activate JNK in transfected cells (Holland, 2016; Gallo, 2002). Why, then, does knockout of DLK causes such striking phenotypes in neurodegeneration models when multiple MLKs are expressed in the same neuronal populations (Yang, 2015)? It is reasoned herein that specific localization and/or post-translational modification of DLK might account for its unique role in neurodegeneration and set out to define such DLK-specific regulation. It was found that DLK is covalently modified by the lipid palmitate, while MLK1-3 are not (FIG. 1B) (Holland, 2016). This process, palmitoylation, can target proteins to specific membranes (Fukata, 2010) and, consistent with this notion, it found that palmitoyl-DLK localizes to axonal transport vesicles in sensory neurons (Holland, 2016). Using lentiviral-mediated knockdown/rescue to replace endogenous DLK with a palmitoyl-site mutant in sensory neurons, it was found that palmitoylation is essential for DLK-dependent signaling following axonal injury (Holland, 2016). Additional findings supported the hypothesis that DLK-dependent injury signals are conveyed on the axonal vesicles (Holland, 2016).

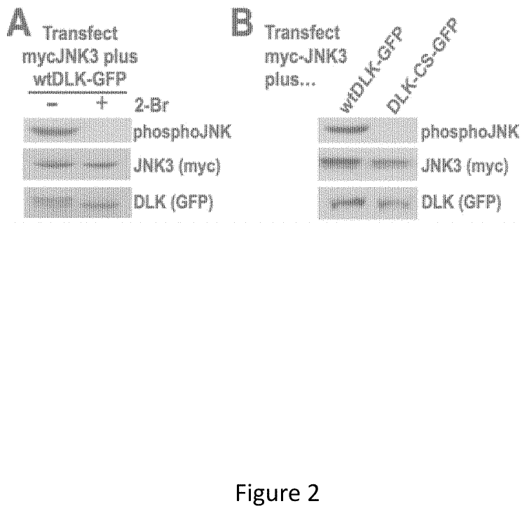

Importantly, though, follow-up experiments revealed that palmitoylation controls not just DLK's localization, but also its ability to activate the JNK pathway (Holland, 2016). GFP-tagged wild type DLK (wtDLK-GFP) potently activated JNK3 in cotransfected HEK293T cells (FIG. 2), but treatment of cells with 2-Bromopalmitate (2-Br, a broad-spectrum palmitoylation inhibitor (Jennings, 2009)), or mutation of DLK's palmitoylation site (Cys127->Ser; “DLK-CS-GFP” mutant) prevented DLK-dependent phosphorylation of JNK3 (FIG. 2) (Holland, 2016).

Experiments were designed herein to assess whether preventing DLK palmitoylation might be an equally effective and/or complementary strategy to directly inhibiting DLK's kinase domain. This approach might also be highly selective because no homologous MAP3Ks, and very few kinases in general, are palmitoylated. Moreover, no palmitoylation inhibitors have been developed therapeutically, so pursuing this approach might reveal a novel class of neuroprotectants. It was thus sought to develop methods to identify inhibitors of DLK palmitoylation.

Palmitoylation Dramatically and Quantifiably Alters DLK Localization

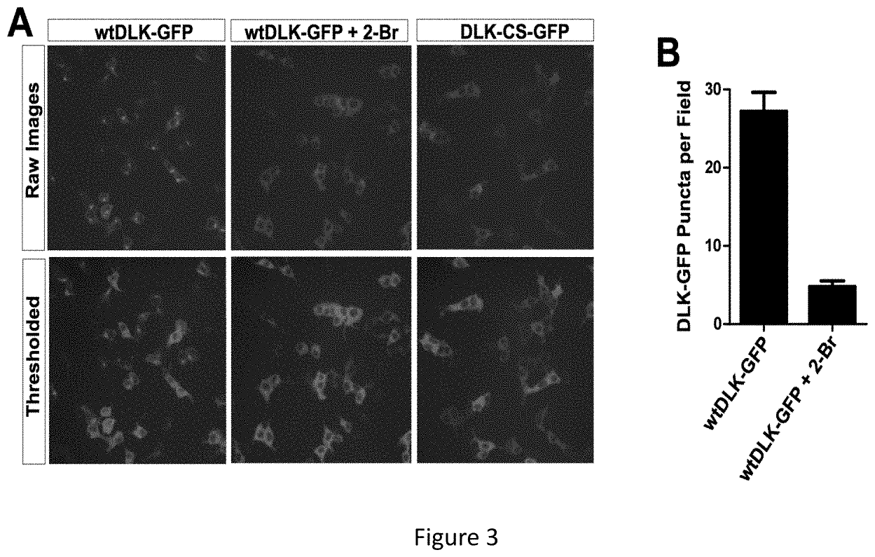

During recent DLK studies (Holland, 2016), a dramatic, palmitoylation-dependent change in DLK localization was observed in heterologous cells. In transfected HEK 293T cells, GFP-tagged wild type DLK (wtDLK-GFP) clearly localizes to intracellular membranes (FIG. 3, top). These membranes are distinct from the vesicles reported in neurons (likely due to different trafficking of DLK in non-neuronal cells) and instead overlap with Golgi markers, likely because many mammalian palmitoyl acyltransferases (PATs) localize to the Golgi (Levy, 2011; Ohno, 2006). Strikingly, either 2-Br treatment or DLK palmitoyl-site mutation completely disrupted DLK-GFP membrane association (FIG. 3). In identically thresholded images, wtDLK-GFP puncta could be readily observed, that were absent in the 2-Br-treated and DLK-CS-GFP images (FIG. 3A, bottom). It was thus reasoned that this palmitoylation-dependent change in DLK localization should be quantifiable using high-content imaging software. Indeed, analysis of the images using an ImageXpress High-Content Image Analysis ‘TransFluor’ module, it was confirmed that 2-Br dramatically alters DLK localization (FIG. 3B). High-content imaging detection is thus well suited to identify novel pharmacological inhibitors of DLK palmitoylation.

An Orthogonal Assay to Confirm Mechanism of Action (MOA) of HTS Hits

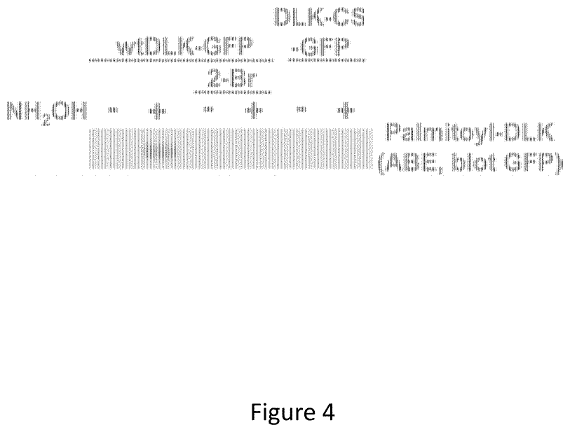

If changes in DLK localization observed by high-content imaging are indeed due to altered DLK palmitoylation then this should also be reflected in biochemical assays of DLK palmitoylation levels. Palmitoylation of numerous proteins, including DLK, has been previously monitored using a non-radioactive palmitoylation assay, Acyl-Biotin Exchange (ABE) (Holland, 2016; Thomas, 2012). ABE uses an exchange of thioester-linked acyl modifications (i.e. palmitoylation), for biotin, with the resultant biotinylated proteins being affinity-purified from cell lysates using neutravidin-conjugated beads. ABE avoids the long incubation times associated with conventional palmitate radiolabeling. WtDLK-GFP was robustly detected in ABE fractions but was absent from controls in which the essential ABE reagent hydroxylamine (NH₂OH) was omitted (FIG. 4). In contrast, no ABE signal was seen in lysates from wtDLK-GFP-expressing cells treated with 2-Br, or in lysates from cells expressing DLK-CS-GFP. Though best suited to medium/low throughput studies, ABE is a robust, orthogonal method to determine MOA of lead compounds identified by high-content imaging.

A Secondary Assay to Determine the Neuroprotective Ability of HTS Hits

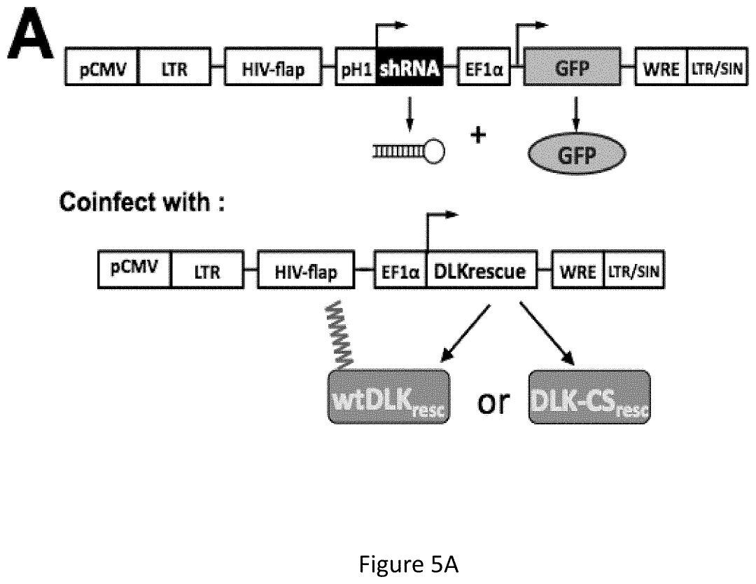

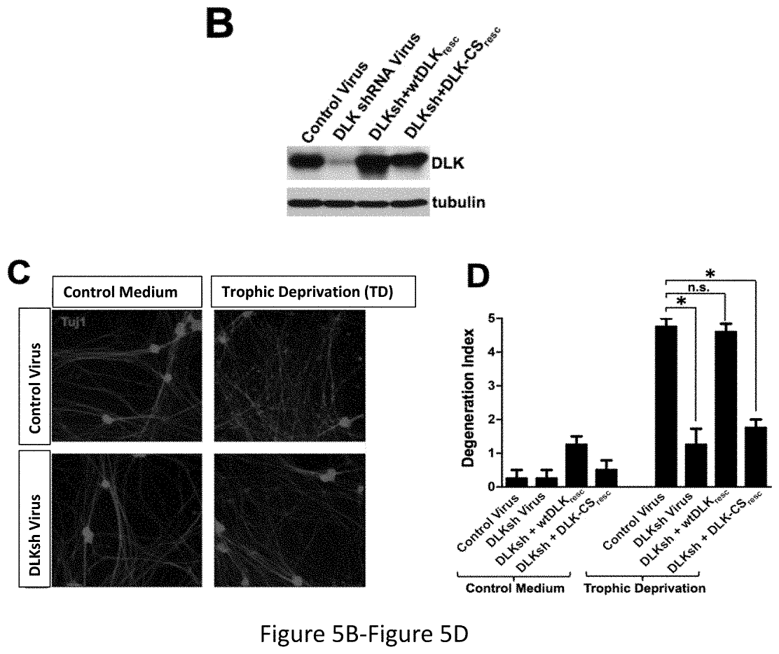

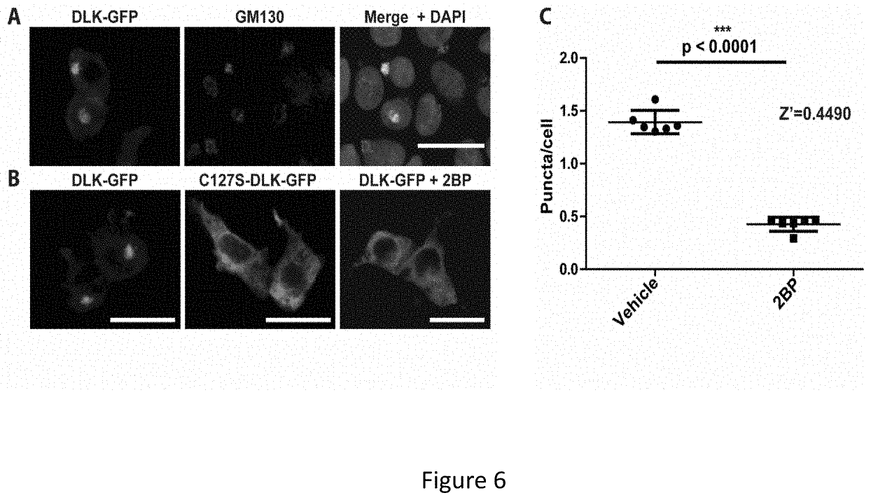

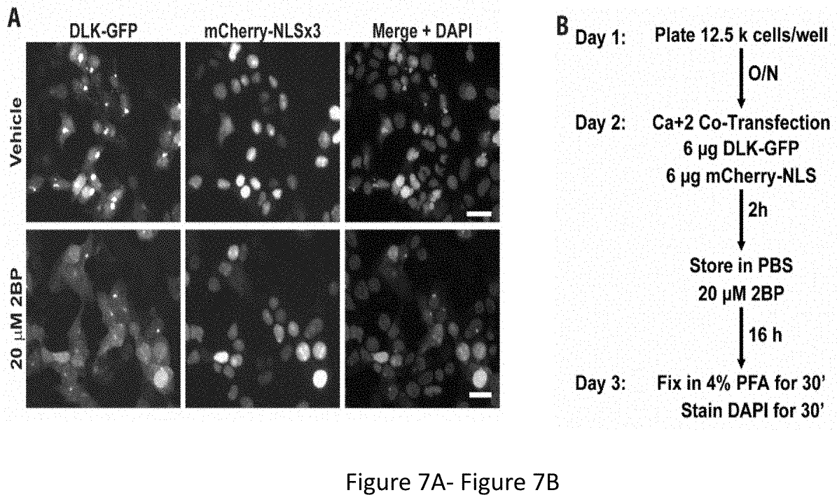

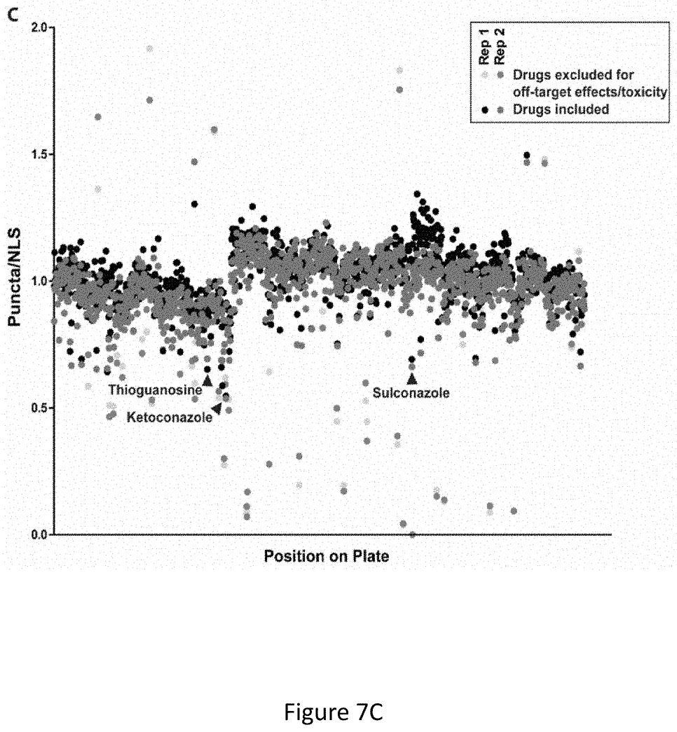

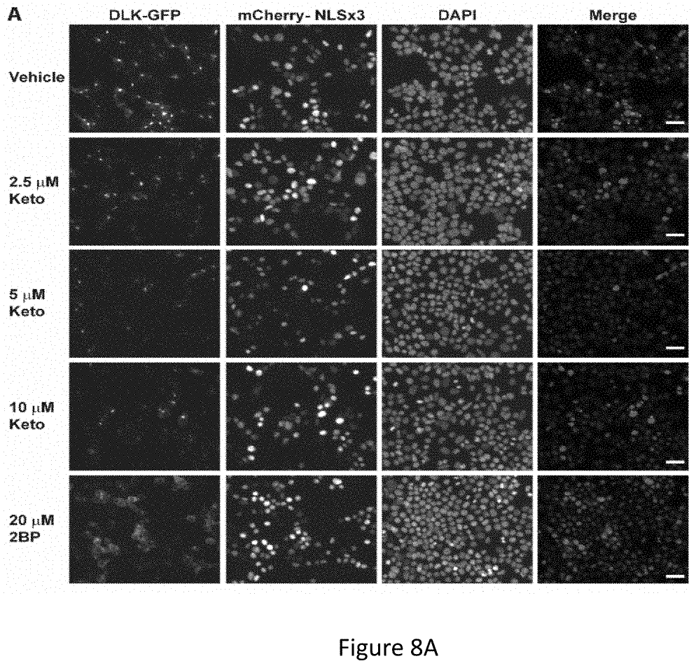

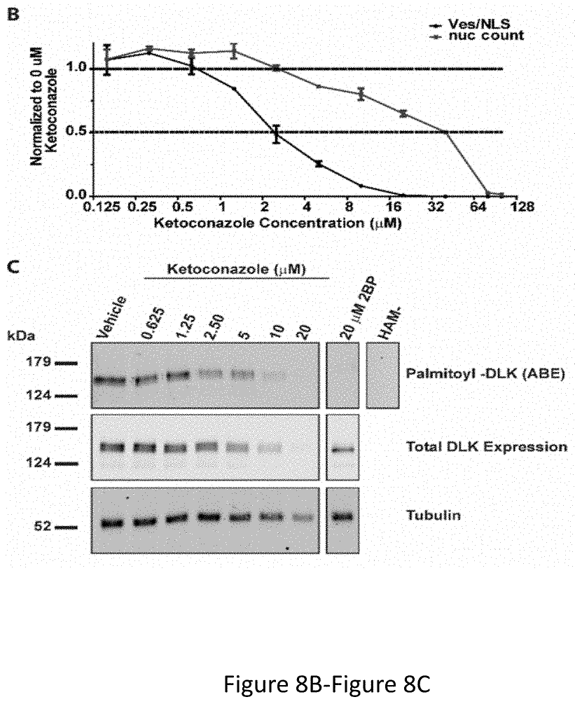

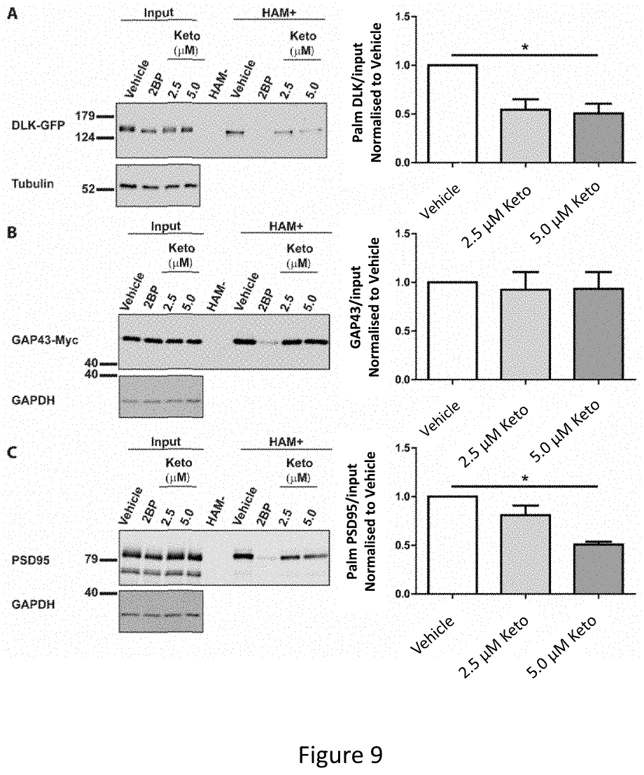

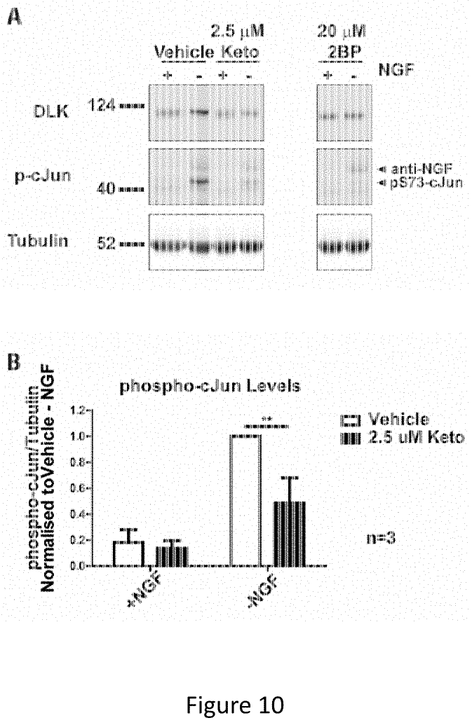

With robust primary and MOA confirmation assays in place, a secondary assay was next optimized in a native, physiologically relevant cell model, which can be used to determine whether any novel inhibitors of DLK palmitoylation are indeed neuroprotective. Trophic Deprivation (TD) induced degeneration in rat sensory neurons was focused upon, which has been recently been used to define pro-degenerative roles for DLK and other proteins (Ghosh, 2011; Simon, 2016; Cosker, 2016). Using shRNA knockdown/rescue, it is revealed that not just DLK, but also its palmitoylation, is critical for TD-induced neurodegeneration (FIG. 5).

A High Content Imaging Screen to Identify Novel Inhibitors of DLK Palmitoylation.

As described herein, the high content imaging screen is optimized. First, the cell-based assay of DLK palmitoylation levels (FIG. 3) is miniaturized to a 384-well format to facilitate HTS. Then, a pilot screen is performed using a Prestwick Chemical Library® of 1200 FDA-approved compounds, followed by a full screen of 20,000 compounds, to identify novel inhibitors of DLK localization.

Miniaturize High-Content Imaging Readout to a 384-Well Plate Format.

Palmitoylation-dependent changes in DLK-GFP localization are readily detectable in HEK293T cells transfected in standard tissue culture plates (FIG. 3), but to facilitate high throughput capacity, this assay is miniaturized to a 384-well format. 2-Br and DLK-CS mutation (known chemical and genetic disruptors of DLK-GFP membrane targeting, respectively) serve as controls to define signal/noise for the assay.

Low passage HEK293T cells are seeded in DMEM/10% FBS at 10,000 cells/well into black μClear 384-well plates, using Biotek MultiFlo dispenser. If necessary, the screens are performed on 96 well plates. Cells are incubated at 37° C., 5% CO₂ overnight, then Lipofectamine 2000 (L2K) is used to transfect wtDLK-GFP cDNA. Four hours post-transfection, medium is replaced with fresh medium containing either 0.1% (v/v) DMSO (compound library solvent control), 0.1% (v/v) EtOH (2-Br solvent control) or 10 mM 2-Br in EtOH. Four hours later, cells are fixed in 4% (w/v) para-formaldehyde and immunostained with anti-GFP antibody and DAPI, as in Figure. 3B (Holland, 2016). DLK membrane association is quantified using ImageXpress as in FIG. 3B. It is predicted that 2-Br treatment will reduce levels of membrane-bound DLK-GFP, as in the initial assay (FIG. 3), and that this reduction will be quantifiable by ImageXpress. In this first step cell density is determined, which is required for optimal signal/noise. Further, Z′, a measure of assay quality that accounts for dynamic range and well-to well variability, is defined (Zhang, 1999). A Z′ value >0.5 is considered to have an assay window and acceptable variability for HTS. To minimize variability and ensure that all volumes can be practically added to wells, cells are initially seeded in 40 ml volume/well and then 10 ml of a master mix containing 0.1 ml L2K/well and 150 ng DNA/well is added. Each parameter is further optimized if required to achieve Z′>0.5.

Perform Prestwick Chemical Library® Pilot Screen to Identify Inhibitors of DLK Palmitoylation

The Prestwick Chemical Library® consists of 1200 FDA-approved compounds. This library, as 10 mM stock solutions in DMSO, is used to perform a pilot screen to identify novel inhibitors of DLK membrane localization. This pilot screen supports validation of the assay and defines a preliminary hit rate. In addition, an advantage of using an FDA-approved library is that any hits would already be approved for human use, thereby offering the opportunity for drug re-purposing.

HEK293T cells are seeded and transfected in 384 well plates with wtDLK-GFP as described elsewhere herein. Four hours later, the 1200 Prestwick Chemical Library compounds are added (1 compound per well, 1200 wells, 4 total plates) at 10 mM final concentration. 0.1% (v/v) DMSO (vehicle control) and 2-Br (positive control) are used for each plate. Four hours later, the cells are fixed and processed for immunostaining and ImageXpress analysis as described elsewhere herein.

It is predicted that inhibitors of DLK palmitoylation reduce levels of membrane-bound DLK-GFP quantified by ImageXpress (FIG. 3B). Compounds that reduce DLK membrane association by >3 standard deviations of the mean (>3×SD), compared to mean of vehicle alone, are validated, as described elsewhere herein.

Perform Primary HTS to Identify Inhibitors of DLK Palmitoylation

After optimization, a full HTS is performed using the Maybridge diversity library, a 20,000 compound set selected from the Maybridge Screening Collection. The library maximizes structural diversity in order to increase the chances of identifying novel pharmacophores for innovative new biological targets. The library obeys Lipinski rules, with all log P values <5 (average log P value=3.2), <5 H-bond donors, <10 H-bond acceptors, <8 rotatable bonds (avg. # of rotatable bonds <5) and MWs <500 (average MW=325). Structural integrity of compounds was confirmed by Maybridge using ¹H-NMR and LC/MS and reconfirmed using LC/MS. All samples were purchased as powders and formulated into 10 mM DMSO stock solutions.

HEK293T cells are seeded and transfected in 384 well plates with wtDLK-GFP cDNA as described elsewhere herein. Four hours post-transfection, the 20,000 Maybridge compounds are added to cells (1 compound per well, 20,000 wells, 63 total plates) at 10 mM final concentration. DMSO (vehicle control) and 2-Br (positive control) are used for each plate. Four hours later the cells are fixed and processed for immunostaining and ImageXpress analysis as described elsewhere herein.

It is predicted that any compounds that inhibit DLK palmitoylation shift DLK distribution from membrane-associated to diffuse, as quantified by ImageXpress (as in FIG. 3B). Compounds that reduce DLK membrane association by >3×SD, compared to mean of vehicle condition are validated as described elsewhere herein.

The Maybridge library has adequate diversity to provide a comprehensive representation of chemical space, as is required for an assay of this type To eliminate compounds that alter DLK localization indirectly by collapsing Golgi stacks (similar to Brefeldin A (Klausner, 1992) the integrity of Golgi markers are confirm as previously described (Thomas, 2012). DAPI staining is used to identify potentially cytotoxic compounds that cause nuclear fragmentation and a direct cytotoxicity assay is performed. Mechanism of Action (MOA) studies eliminate other compounds that affect DLK localization indirectly.

Finally, it is noted that palmitoylation is a reversible modification and that turnover of palmitate on DLK is very rapid (half-life <1 hour (Holland, 2016)). This finding increases the likelihood that DLK palmitoylation inhibitors are effective in the cell-based assay and also raises the probability that such inhibitors can be effectively therapeutically.

Target Validation of HTS Hits and Determine Whether they Act as Novel Neuroprotectants

Experiments presented herein validate and prioritize HTS hits identified in the screening assays described above. A replicate study is performed in triplicate from new samples of each hit compound, followed by a cytotoxicity assay to eliminate false positives. Potency of remaining hits is determined; MOA is confirmed in an orthogonal biochemical assay and finally assays in cultured neurons are performed to determine the neuroprotective ability of validated hits.

Confirm Primary Screen Results in Replicate Assay.

First, to ensure that all ‘hits’ that are pursued are robust and reproducible, each compound that inhibited DLK membrane association by >3×SD in the primary screen is re-assayed using the same conditions described above.

All compounds that showed >50% inhibition of DLK membrane association are re-purchased, and DLK localization assays are repeated with fresh stocks in triplicate. Compounds that are bonafide disruptors of DLK membrane association act as demonstrated in the initial screen. This verification step allows for the elimination of technical false-positives before progressing further.

Eliminate Cytotoxic False Positive Hits

Cytotoxicity assays can eliminate false positives in cell-based HTS studies. CellTiter-Glo® assays are performed to determine the number of viable cells in culture based on cellular ATP levels. This one-step assay is designed for multiwell formats and is ideal for automated HTS studies.

For all compounds that inhibited DLK membrane association >3 SD, plates are processed for CellTiter-Glo® Assay. Luminescence is quantified using a Perkin Elmer Envision Plate reader. Compounds that reduce ATP levels by >3×SD of the mean, compared to vehicle treated cells, are discarded due to likely cytotoxicity. Compounds that reduce ATP levels by >2×SD of the mean when used at 10 mM are included in potency assays, but only pursued in mechanism of action studies if IC₅₀<1 mM.

Determine Potency of Compounds that Prevent DLK Membrane Association in Primary Screen.

All experiments to this point have used a compound concentration of 10 mM. Experiments presented herein identify the most potent hits by determining half-maximal inhibitory concentrations (IC₅₀) over a 10-point dose response curve.

HEK293T cells are seeded in 384 well plates and transfected with wtDLK-GFP cDNA as described elsewhere herein. For any compound defined as non-cytotoxic, compound is added to cells over a 10-point range (100 mM-1 nM, plus vehicle control) in duplicate. IC₅₀ for disruption of DLK membrane association for each compound is determined. In this step all hit compounds from the cytotoxic assays that pass cut-off criteria is pursued. However, compounds with submicromolar IC₅₀ values are prioritized in subsequent steps.

Determine Mechanism of Action of HTS Hits Using an Orthogonal Biochemical Assay.

Experiments presented herein test the ability of all non-cytotixic hits to 2 to reduce DLK palmitoylation in biochemical assays. HEK293T cells are seeded in 10 cm dishes and transfected with wtDLK-GFP cDNA as described elsewhere herein. Assays are performed in triplicate. Four hour post-transfection, a separate compound (10 mM final concentration) is added to each set of triplicate dishes. 2-Br is used as positive control. Cells are lysed 4 hours later and processed for ABE. Western blots are used to detect palmitoyl-DLK in ABE fractions and total DLK in cell lysates. Compounds that are bonafide inhibitors of DLK palmitoylation reduce levels of palmitoyl-DLK in ABE fractions without affecting total DLK expression level in lysates. ABE is widely used to monitor protein palmitoylation (e.g. Wan, 2007), but other palmitoylation assays e.g. ³H-palmitate radiolabeling (Thomas, 2012; Hayashi, 2009) to further verify MOA of compounds of interest. ABE was previously used to assay all 23 mammalian palmitoyl acyltransferases (PATs) (Holland, 2016), which, including controls, required parallel processing of >30 samples. It is therefore not unrealistic to scale up ABE assays to process the max. number of samples (100) that to follow up from the primary screen.

Novel DLK Palmitoylation Inhibitors Prevent TD-Induced Degeneration of Sensory Neurons

Trophic Deprivation (TD) of sensory neurons is widely used to identify regulators of neurodegeneration and requires not just DLK, but also its palmitoylation (Ghosh, 2011; Simon, 2016; Cosker, 2016; Mok, 2009) (FIG. 5D). Thus, it is examined whether identified inhibitors of DLK palmitoylation prevent TD-induced degeneration.

Embryonic sensory neurons are plated in NGF-containing medium, as in FIG. 5. At 5 days in vitro (DIV) medium is replaced with NGF-free medium containing anti-NGF antibody, plus identified compounds or DMSO vehicle. Neurons are fixed 24 hours later, immunostained with anti-Tuj1 antibody, and the extent of neurodegeneration is scored as in FIG. 5. The assay is repeated 3 times, from at least 2 different dissections of neurons. Compounds that are neuroprotective reduce the extent of TD-induced degeneration.

The experiments described herein identify 3 categories of DLK palmitoylation inhibitors:

(i) Broad spectrum palmitoylation inhibitors that act similarly to 2-Br. Such hits will be useful to basic research on palmitoylation, which relies almost exclusively on 2-Br.

(ii) Inhibitors of specific PAT(s) that palmitoylate DLK. Hits of this type have considerable therapeutic potential, though care should be taken if the PAT(s) palmitoylates multiple substrates. Specific PAT inhibitors will also greatly aid palmitoylation research, for which no such compounds are available, and also treatments of other disease conditions (e.g. certain cancers in which PATs are upregulated or hyper-activated (Koryka, 2012)).

(iii) Allosteric modulators of DLK conformation that prevent its palmitoylation. These compounds are highly likely to be selective because DLK's palmitoylation site is not conserved in other kinases (Holland, 2016). Moreover, the finding that palmitoylation regulates DLK protein-protein interactions (Holland, 2016) strongly suggests that the conformation of the region around DLK's palmitoyl-site is flexible and could thus be altered by a small molecule. Such compounds have considerable therapeutic potential.

Importantly, because inhibition of palmitoylation has not been pursued as a neuroprotective strategy, many hits identified in the described screens may well be novel compounds of different classes to those in current use. Thus, the experiments presented herein may open up entirely new therapeutic avenues.

The novel approach described herein holds considerable therapeutic promise. In addition, chemi-informatics is used to cluster hits and identify common functional moieties. Key next steps to determine cell permeability, metabolic liability (possible reactive metabolite formation) and other properties of lead compounds in order to further prioritize them, and to optimize validated hits via medicinal chemistry refinement/derivatization.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of identifying a modulator of palmitoylation comprising:
a) administering the at least one test compound to a cell modified to comprise DLK tagged with a detectable label;
b) detecting DLK membrane association; and
c) identifying the test compound as a modulator of palmitoylation when the test compound alters DLK membrane association.

2. The method of claim 1, wherein the test compound is identified as an inhibitor when DLK membrane association is reduced compared to control conditions where a test compound is not administered.

3. The method of claim 2, wherein the identified inhibitor is at least one selected from the group consisting of: a broad palmitoylation inhibitor, a specific inhibitor of one or more palmito acyltransferase (PAT), and a modulator of DLK conformation.

4. The method of claim 1, wherein the method comprises the use of a high throughput screen and wherein the test compound is from a library of test compounds.

5. The method of claim 1, wherein DLK membrane association is detected by quantifying puncta of the detectable label.

6. The method of claim 1, wherein the method further comprises conducting a cytotoxicity assay on an identified inhibitor of palmitoylation.

7. The method of claim 1, wherein the method further comprises conducting an assay to evaluate the effect of the identified inhibitor of palmitoylation on neurodegeneration.

8. The method of claim 2, wherein the identified inhibitor of palmitoylation is neuroprotective.

9. A method of treating a disease or disorder in a subject in need thereof, comprising administering an effective amount of a modulator of palmitoylation identified by the method of claim 1.

10. The method of claim 9, wherein the disease or disorder is associated with neurodegeneration.
